(19) United States
(12) Patent Application Publication
(10) Pub. No.: US 2023/0268096 A1
(43) Pub. Date: Aug. 24, 2023

(54) SYSTEMS, DEVICES, AND METHODS FOR MULTI-DIRECTIONAL DIPOLE MAGNETS AND COMPACT BEAM SYSTEMS

(71) Applicant: TAE TECHNOLOGIES, INC., Foothill Ranch, CA (US)

(72) Inventors: Greg Snitchler, Foothill Ranch, CA (US); Ken Yoshiki Franzen, Irvine, CA (US); Charles Lee, Irvine, CA (US); Jedediah Styron, Mission Viejo, CA (US); Vijay Patel, Eastvale, CA (US); Shayar Ziaei, Ladera Ranch, CA (US)

(21) Appl. No.: 18/174,467

(22) Filed: Feb. 24, 2023

Related U.S. Application Data
(60) Provisional application No. 63/313,622, filed on Feb. 24, 2022.
(60) Provisional application No. 63/478,763, filed on Jan. 6, 2023.

(51) Int. Cl. G21K 1/093; H05H 3/06; H01F 7/20; A61N 5/10

(52) U.S. Cl. CPC G21K 1/093; H05H 3/06; H01F 7/20; A61N 5/1078; A61N 2005/109; A61N 2005/1094

(57) ABSTRACT

Embodiments of systems, devices, and methods relate to controlling beams for use in beam systems. An example method of controlling a travel path of a beam includes propagating a beam along a first path from an entry point of a dipole magnet through a non-gradient portion of the dipole magnet until the beam bends toward a first beam travel path of multiple beam travel paths of the dipole magnet. The example method further includes propagating the beam along the first beam travel path through a gradient portion of the dipole magnet to focus the beam for propagation to a downstream target. Embodiments further permit a compact beam system such that a series of magnets can be used to create a path that accommodates shielding to minimize the footprint of the beam system for facilities that may not otherwise support large systems due to space and safety constraints.

### CROSS-REFERENCE TO RELATED APPLICATIONS

The present application claims priority to U.S. Provisional Application Ser. No. 63/313,622, titled “SYSTEMS, DEVICES, AND METHODS FOR MULTI-DIRECTIONAL DIPOLE MAGNETS”, filed Feb. 24, 2022, and to U.S. Provisional Application Ser. No. 63/478,763, titled “SYSTEMS, DEVICES, AND METHOD FOR USING MULTI-DIRECTIONAL DIPOLE MAGNETS FOR COMPACT BEAM SYSTEMS,” filed Jan. 6, 2023, the contents of both of which are incorporated herein in their entirety for any purpose.

### FIELD

The subject matter described herein relates generally to systems, devices, and methods of controlling beams for use in beam systems and reducing beam system footprints.

### BACKGROUND

Boron neutron capture therapy (BNCT) is a modality of treatment of various types of cancers. Neutron beam systems used for BNCT typically include a target device that, when impacted by a beam of energetic protons, produces a neutron beam capable of treating cancerous tumors, including those associated with some of the most aggressive and difficult to treat cancer types. BNCT selectively aims to treat tumor cells while sparing the normal cells using a boron compound. A substance that contains boron is injected into a blood vessel, and the boron collects in tumor cells. The patient then receives radiation therapy with neutrons (e.g., in the form of a neutron beam). The neutrons react with the boron to kill the tumor cells without harming normal cells. Prolonged clinical research has proven that a beam of neutrons with an energy spectrum within 3-30kiloelectronvolts (keV) is preferable to achieve a more efficient cancer treatment while decreasing a radiation load on a patient. This energy spectrum or range is frequently referred to as epithermal.

The process of generating epithermal neutrons (e.g., epithermal neutron beams), such as those used to treat various types of cancer, can require a nuclear reaction of protons (e.g., a proton beam) with either a Beryllium or Lithium (e.g. a Beryllium or Lithium target). This nuclear reaction created by the collision of protons onto the target can create neutrons within the desired epithermal energy spectrum. In order to reach the desired energy spectrum for the produced neutron beam and to maximize the number of neutrons produced, controllable beam optics are needed to ensure that the proton beam cross-section and phase space are controlled throughout the beamline such that the proton beam can be scanned onto the target with reasonable uniformity.

Conventional approaches can use a series of magnets to direct and/or focus a beam toward a target or other desired location. However, such conventional methods typically utilize a set of dipole magnets to direct a beam towards a desired direction and, separately, use a set of quadrupole magnets to focus the beam. This can result in a long beamline with a complex focusing scheme. Further, conventional dipole magnets have substantially flat pole faces and have not conventionally been used for beam focusing. This is mainly due to a conventional dipole magnet's inability to focus a beam in both transverse directions and tendency to overfocus the beam in in one transverse direction, which can result in beam collapse.

Moreover, in radiation oncology, there has been an emphasis on large facilities that support multiple treatment rooms and can easily support the additional space required to provide radiation shielding for accelerator rooms and the treatment rooms. BNCT based on tandem accelerators generally have a small footprint, but size and safety constraints continue to present drawbacks associated with buildings or spaces that are unable to accommodate the systems. In particular, it is difficult to shield critical accelerator components that have direct line of sight to the lithium target.

For these and other reasons, a need exists for improved, efficient, and compact systems, devices, and methods capable of controlling beams in a beam system using magnets and neutronics shielding walls.

### SUMMARY

Embodiments of systems, devices, and methods relate to directing and focusing a charged particle beam. An example method of controlling a travel path of a beam includes propagating a beam along a first path from an entry point of a dipole magnet through a non-gradient portion of the dipole magnet until the beam bends toward a first beam travel path of multiple beam travel paths of the dipole magnet. The example method further includes propagating the beam along the first beam travel path through a gradient portion of the dipole magnet to focus the beam for propagation to a downstream target.

Embodiments of systems, devices, and methods further relate to steering a charged particle beam from an accelerator through neutron shielding walls to a target location surrounded by a beam shaping assembly that is integrated into a neutronics shielding wall. Embodiments permit a compact beam system (e.g., for use in boron neutron capture therapy) such that a series of magnets can be used to create a path that accommodates shielding to minimize the footprint of the beam system for facilities that may not otherwise support large systems due to space and safety constraints.

Other systems, devices, methods, features and advantages of the subject matter described herein will be or will become apparent to one with skill in the art upon examination of the following figures and detailed description. It is intended that all such additional systems, methods, features and advantages be included within this description, be within the scope of the subject matter described herein and be protected by the accompanying claims. In no way should the features of the example embodiments be construed as limiting the appended claims, absent express recitation of those features in the claims.

### BRIEF DESCRIPTION OF FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1A is a schematic diagram of an example embodiment of a beam system usable with embodiments of the present disclosure.

FIG. 1B is a schematic diagram of an example beam system usable in boron neutron capture therapy (BNCT).

FIG. 1C is a schematic diagram of a portion of an example beam system usable with embodiments of the present disclosure.

FIG. 1D is a schematic diagram of a portion of an example beam system usable with embodiments of the present disclosure.

FIGS. 2A, 2B, 2C, 2D, 2E, and 2F illustrate an example beam trajectory within a dipole magnet of a beam system, in accordance with embodiments of the present disclosure.

FIG. 3 illustrates an example beam system usable with embodiments of the present disclosure.

FIG. 4 illustrates an example beam injection entryway example usable in beam guidance, in accordance with embodiments of the present disclosure.

FIG. 5 illustrates an example beam system with two beam travel channels usable with embodiments of the present disclosure.

FIG. 6 illustrates magnetic field distribution at the midplane of the bidirectional dipole magnets, in accordance with embodiments of the present disclosure.

FIGS. 7A-7B illustrate an example of a beam envelope in both transverse directions in accordance with a bidirectional dipole magnet having only a non-gradient portion.

FIGS. 8A-8B illustrate an example of a beam envelope in both transverse directions in accordance with a bidirectional dipole magnet having a gradient portion.

FIG. 9 illustrates an example beam system usable with embodiments of the present disclosure.

FIG. 10 illustrates an example beam system usable with embodiments of the present disclosure.

FIG. 11 illustrates an example beam system usable with embodiments of the present disclosure.

FIG. 12 illustrates an example beam system usable with embodiments of the present disclosure.

FIG. 13 illustrates an example beam system usable with embodiments of the present disclosure.

FIG. 14 illustrates an example beam system usable with embodiments of the present disclosure.

FIG. 15 illustrates an example beam system usable with embodiments of the present disclosure.

FIG. 16 illustrates an example beam system usable with embodiments of the present disclosure.

FIG. 17 illustrates an example beam system usable with embodiments of the present disclosure.

FIG. 18 illustrates an example beam system usable with embodiments of the present disclosure.

FIG. 19 illustrates an example beam system usable with embodiments of the present disclosure.

FIG. 20 illustrates an example beam system usable with embodiments of the present disclosure.

FIG. 21 illustrates an example beam system usable with embodiments of the present disclosure.

FIG. 22 illustrates an example beam system usable with embodiments of the present disclosure.

FIGS. 23A-23B show an example profile of a beam envelope for a beam within a beam system using a pair of dipole magnets, in accordance with embodiments herein.

FIGS. 24A-24B show an example profile of a beam envelope for a beam within a beam system using a pair of quadrupole magnets, in accordance with embodiments herein.

### DETAILED DESCRIPTION

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

The term “particle” is used broadly herein and, unless otherwise limited, can be used to describe an electron, a proton (or H+ ion), or neutron, as well as species having more than one electron, proton, and/or neutron (e.g., other ions, atoms, and molecules).

Example embodiments of systems, devices, and methods are described herein for a beam guidance system usable with a beam system. The embodiments described herein can be used with any type of beam, including beams formed from various particle types.

For ease of description, many embodiments described herein will be done so in the context of a proton beam used to form a neutron beam for use in BCNT, although the embodiments are not limited to just proton beams, neutron beams, nor BNCT applications.

Embodiments described herein can streamline a beamline in a beam system and simplify focusing processes used in accordance with neutron beams. That is, embodiments of the present disclosure overcome several disadvantages associated with limitations introduced by equipment in the context of beam systems. A beam system must both direct a proton beam to a target and properly focus the proton beam to ensure efficient production of neutrons for a neutron beam produced during the collision of a proton beam and a target. In order to do so, a beam system can include dipole magnets (e.g., bending magnets) to direct a beam and quadrupole magnets to focus the beam prior to the collision with the target.

Advantageously, embodiments of the present disclosure permit direction of a beam and focusing of said beam by utilizing a dipole magnet with a non-gradient portion as well as a gradient portion. Accordingly, the non-gradient portion of the dipole magnet can direct the beam in a desired direction and the gradient portion of the dipole magnet can focus the beam in both transverse directions. The beam can be focused such that the final beam shape can be scanned onto a target substantially uniformly. As such, this removes the need for an additional quadrupole magnet to focus the beam and allows the beam system to become more compact and simplified.

In radiation therapy, there has been an emphasis on large facilities that support multiple treatment rooms and that can easily support the additional space required to provide radiation shielding for accelerator rooms and the treatment rooms. BNCT based on tandem accelerators generally have a small footprint as compared to other radiation therapy systems, however drawbacks still exist with respect to size and safety constraints. For example, it is difficult to shield critical accelerator components that have direct line of sight to the lithium target. Moreover, the ability to steer a beam in multiple directions such that multiple treatment rooms can be safely supported is still of interest.

Embodiments herein overcome the aforementioned drawbacks and more by employing series of magnets to steer the beam from the accelerator through neutron shielding walls to a target location surrounded by a beam shaping assembly that is integrated into a neutronic shielding wall. Accordingly, a compact BNCT system is provided such that a correct series of bending magnets create a labyrinth path that accommodates shielding to minimize the footprint of the BNCT for medical facilities that do not support large systems. Embodiments herein employ bending magnets to achieve maximum radiation shielding in the lowest machine footprint including treatment rooms, HEBL, accelerator room, and target storage rooms.

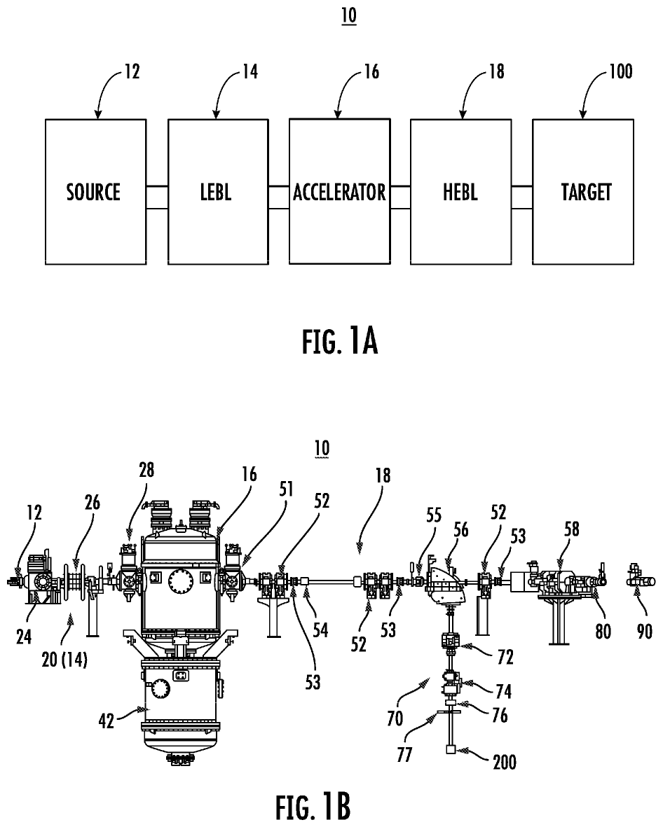

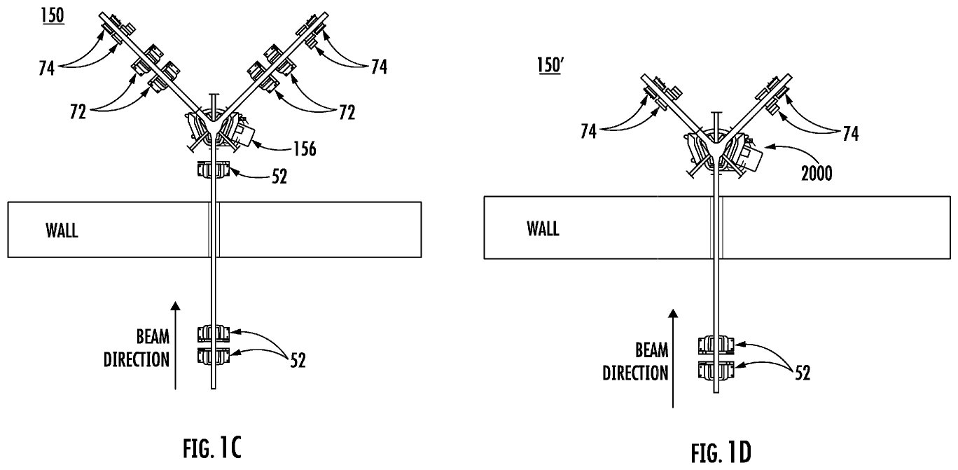

FIG. 1A is a schematic diagram of an example embodiment of a beam system 10 usable with embodiments of the present disclosure. In FIG. 1A, beam system 10 includes a source 12, a low-energy beamline (LEBL) 14, an accelerator 16 coupled to the low-energy beamline (LEBL) 14, and a high-energy beamline (HEBL) 16 extending from the accelerator 16 to a target 100. LEBL 14 is configured to transport a beam from source 12 to an input of accelerator 16, which in turn is configured to produce a beam by accelerating the beam transported by LEBL 14. HEBL 18 transfers the beam from an output of accelerator 16 to target 100. Target 100 can be a structure configured to produce a desired result in response to the stimulus applied by the incident beam, or can modify the nature of the beam. Target 100 can be a component of system 10 or can be a workpiece that is conditioned or manufactured, at least in part, by system 10.

FIG. 1B is a schematic diagram illustrating another example embodiment of a neutron beam system 10 usable in boron neutron capture therapy (BNCT). Here, source 12 is an ion source and accelerator 16 is a tandem accelerator. Neutron beam system 10 includes a pre-accelerator system 20, serving as a charged particle beam injector, high voltage (HV) tandem accelerator 16 coupled to pre-accelerator system 20, and HEBL 18 extending from tandem accelerator 16 to a neutron target assembly 200 housing target 100 (not shown). In this embodiment target 100 is configured to generate neutrons in response to impact by protons of a sufficient energy, and can be referred to as a neutron generation target. Neutron beam system 10 as well as pre-accelerator system 20 can also be used for other applications such as those other examples described herein, and is not limited to BNCT.

Pre-accelerator system 20 is configured to transport the ion beam from ion source 12 to the input (e.g., an input aperture) of tandem accelerator 16, and thus also acts as LEBL 14. Tandem accelerator 16, which is powered by a high voltage power supply 42 coupled thereto, can produce a proton beam with an energy generally equal to twice the voltage applied to the accelerating electrodes positioned within accelerator 16. The energy level of the proton beam can be achieved by accelerating the beam of negative hydrogen ions from the input of accelerator 16 to the innermost high-potential electrode, stripping two electrons from each ion, and then accelerating the resulting protons downstream by the same applied voltage.

HEBL 18 can transfer the proton beam from the output of accelerator 16 to the target within neutron target assembly 200 positioned at the end of a branch 70 of the beamline extending into a patient treatment room. System 10 can be configured to direct the proton beam to any number of one or more targets and associated treatment areas. In this embodiment, the HEBL 18 includes three branches 70, 80 and 90 that can extend into three different patient treatment rooms, where each branch can terminate in a target assembly 200 and downstream beam shaping apparatus (not shown). HEBL 18 can include a pump chamber 51, quadrupole magnets 52 and 72 to prevent de-focusing of the beam, dipole or bending magnets 56 and 58 to steer the beam into treatment rooms, beam correctors 53, diagnostics such as current monitors 54 and 76, a fast beam position monitor 55 section, and a scanning magnet 74. Magnet 58 can divert or steer the beam in separate horizontal directions associated with branches 80 and 90.

The design of HEBL 18 depends on the configuration of the treatment facility (e.g., a single-story configuration of a treatment facility, a two-story configuration of a treatment facility, and the like). The beam can be delivered to target assembly (e.g., positioned near a treatment room) 200 with the use of bending magnet 56. Quadrupole magnets 72 can be included to then focus the beam to a certain size at the target. Then, the beam passes one or more scanning magnets 74, which provides lateral movement of the beam onto the target surface in a desired pattern (e.g., spiral, curved, stepped in rows and columns, combinations thereof, and others). The beam lateral movement can help achieve smooth and even time-averaged distribution of the proton beam on the lithium target, preventing overheating and making the neutron generation as uniform as possible within the lithium layer.

After entering scanning magnets 74, the beam can be delivered into a current monitor 76, which measures beam current. Target assembly 200 can be physically separated from the HEBL volume with a gate valve 77. The main function of the gate valve is separation of the vacuum volume of the beamline from the target while loading the target and/or exchanging a used target for a new one. In embodiments, the beam is not bent by 90 degrees by a bending magnet 56, it rather goes straight to the right of FIG. 1B, then enters quadrupole magnets 52, which are located in the horizontal beamline. The beam could be subsequently bent by another bending magnet 58 to a needed angle, depending on the building and room configuration.

Otherwise, bending magnet 58 could be replaced with a Y-shaped magnet in order to split the beamline into two directions for two different treatment rooms located on the same floor.

Given the complexities of the beam system 10, it may be advantageous for a beam system 10 to be shorter in length, smaller in overall footprint, and/or require a less complex focusing scheme. A more compact and less complex beam system 10 may require less time to properly adjust the components of the beam system 10 and may also require less maintenance to maintain the components of the beam system 10. Embodiments herein provide a beam system 10 to include a dipole magnet with a non-gradient portion as well as a gradient portion, such that the dipole magnet may both direct and focus a beam. Thus, the need for one or more quadrupole magnets to focus the beam may be eliminated, allowing the beam system 10 to become more compact and less complex.

FIG. 1C is a schematic diagram illustrating a portion 150 of a beam system 10. In FIG. 1C, portion 150 of beam system 10 uses one or more quadrupole magnets 52 to focus a beam before the beam enters the dipole or bending magnet 156. The dipole or bending magnet 156 is then used to direct the beam in a particular direction. In this case, the beam may be directed on a first (e.g., appearing to the left in FIG. 1C) beam path or a second (e.g., appearing to the right in FIG. 1C) beam path. Whether the beam is directed on a first or second beam path, the beam passes through one or more quadrupole magnets 72 before passing through one or more scanning magnets 74 and then delivered to a target assembly (not shown in FIG. 1C).

FIG. 1D is a schematic diagram illustrating an alternative portion 150′ of a beam system 10. In comparison to FIG. 1C, the alternative portion 150′ of beam system 10 in FIG. 1D uses a dipole magnet 2000 in accordance with embodiments of the present disclosure (e.g., dipole magnet 2000 can include a non-gradient portion as well as a gradient portion). The alternative portion 150′ of beam system 10 (e.g., which employs the dipole magnet 2000) passes a beam through one or more quadrupole magnets 52 prior to the beam entering dipole magnet 2000. The beam can then pass through dipole magnet 2000, where it can become focused to a certain size for a target and also be steered in a particular direction by dipole magnet 2000. As dipole magnet 2000 advantageously focuses the beam to a desired beam size, the beamline and overall footprint of the beam system 10 may be shortened or reduced such that the one or more quadrupole magnets 72 are no longer needed (e.g., in comparison to FIG. 1C) or can be significantly reduced in size. Moreover, the current and field of the magnet 2000 can be adjusted to accommodate a range of desired beam energies in accordance with system parameters or application. As such, the beam can pass from dipole magnet 2000 to scanning magnets 74, and the beam can then be delivered to a target assembly (not shown in FIG. 1D). This reduction in beamline footprint and complexity due to eliminating one or more quadrupole magnets provides a number of advantages, including fewer components to maintain and protect against beam misalignment, thus reducing space, cost, labor resources, maintenance time and resources, downtime, and safety risks.

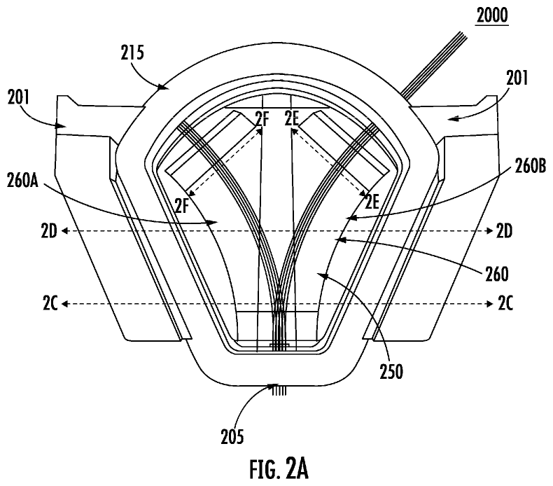

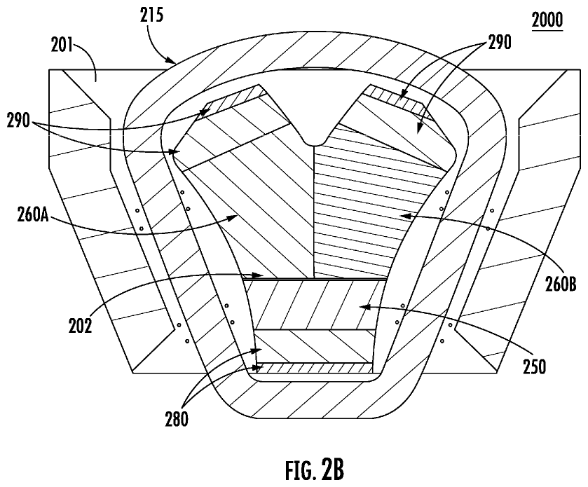

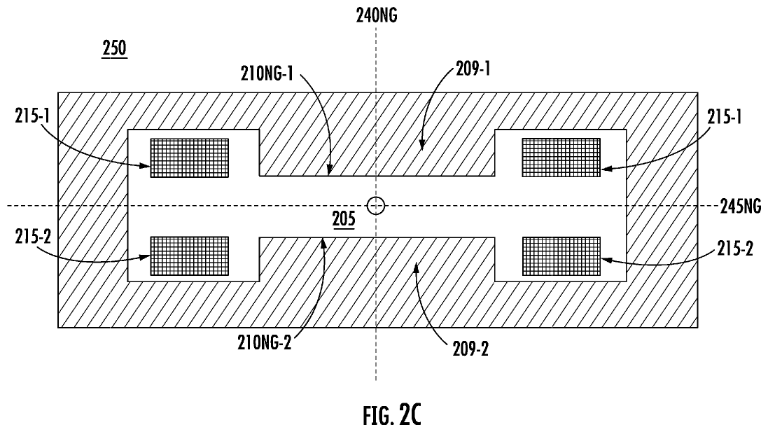

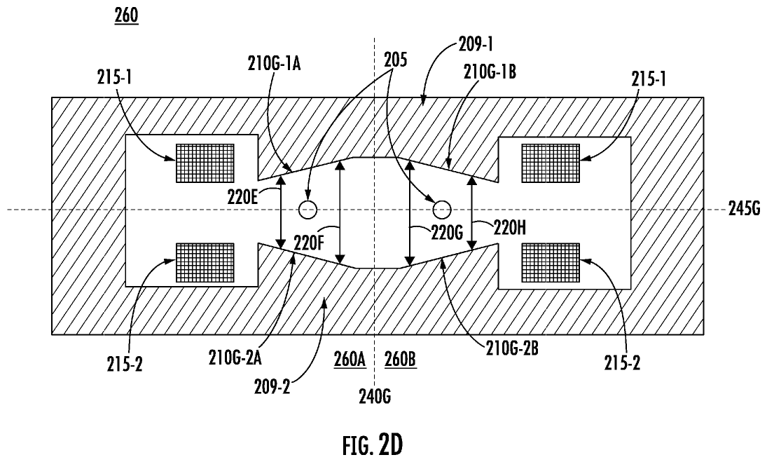

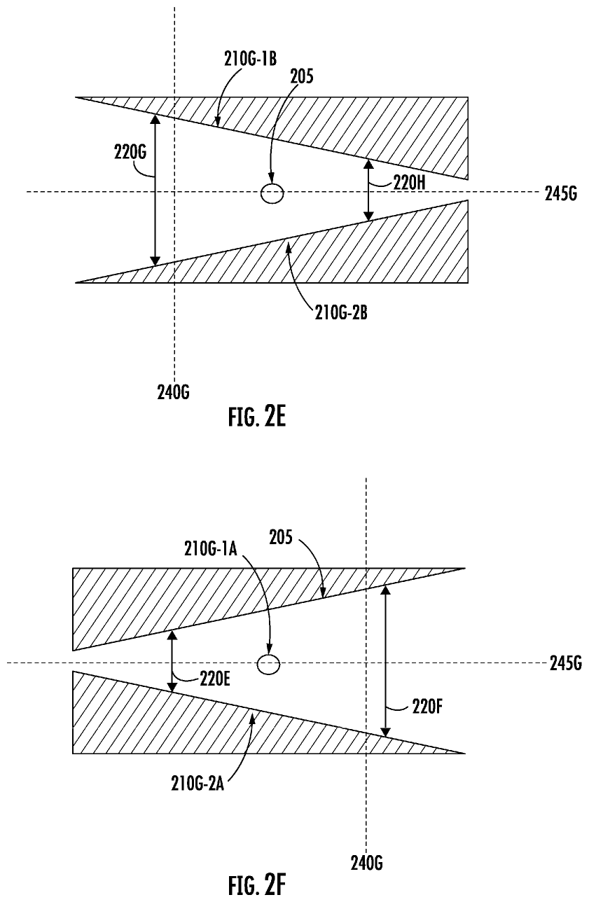

FIGS. 2A, 2B, 2C, 2D, 2E, and 2F illustrate an example embodiment of dipole magnet 2000 with example beam trajectories therethrough. Magnet 2000 can be part of a beam system 10 as described herein. Magnet 2000 includes a core structure, or yoke, 201 having a first pole 209-1 (having a first pole face 210-1 (e.g., having surfaces 210NG-1, 210G-1A, 210G-1B)) and a second pole 209-2 (having a second pole face 210-2 (e.g., having surfaces 210NG-2, 210G-2A, 210G-2B)). A first annular winding, or coil, 215-1 is located around the periphery of first pole 209-1, and a second annular winding, or coil, 215-2 is located around the periphery of second pole 209-2. For ease of discussion, first pole 209-1, pole face 210-1, and winding 215-1 will be referred to as located on top and second pole 209-2, pole face 210-2, and winding 215-2 will be referred to as located on bottom, according to the orientation depicted in FIGS. 2C-2F.

As shown in FIG. 2A, a beam 205 is injected between top pole face 210-1 (not shown in FIG. 2A) and bottom pole face 210-2 (e.g., shown below or underneath the beam 205 in FIG. 2A) of dipole magnet 2000. Each of the top pole face 210-1 and bottom pole face 210-2 can include a gradient pole face portion (e.g., corresponding to gradient portion 260, comprised of 260A (e.g., having surfaces 210G-1A and 210G-2A) and 260B (e.g., having surfaces 210G-1B and 210G-2B)) and non-gradient pole face portion (e.g., corresponding to non-gradient portion 250). The dipole magnet 2000 further includes a pair of windings 215 (e.g., 215-1 and 215-2), which may be used in part to induce a magnetic field between the top pole face 210-1 and bottom pole face 210-2. Core structure 201 can be a laminated magnetic core structure, which can be parallel to a magnetic field of the non-gradient portion 250.

When beam 205 is injected into the dipole magnet 2000, the beam 205 first encounters a non-gradient portion 250 formed by non-gradient portions (not shown) of each of the top pole face 210-1 and bottom pole face 210-2. The non-gradient portion 250 of dipole magnet 2000 can be formed by a non-gradient top pole face 210NG-1 (not shown in FIGS. 2A or 2B, but shown in FIG. 2C) and non-gradient bottom pole face 210NG-2 (not shown in FIGS. 2A or 2B, but shown in FIG. 2C). Each of non-gradient top pole face 210NG-1 and non-gradient bottom pole face 210NG-2 can be planar or flat (e.g., or substantially planar or substantially flat) and parallel to one another.

Shown in FIG. 2B, prior to the non-gradient portion 250 of the dipole magnet 2000, one or more chamfered edges 280 can be included on the top pole face 210-1 and bottom pole face 210-2, respectively, and extend along an edge that is perpendicular to a direction of travel of an injected beam. After the gradient portion 260 of the dipole magnet, prior to an exit of the dipole magnet 2000, one or more additional chamfered edges 290 can be included on the top pole face 210-1 and bottom pole face 210-2. Chamfered edges 280 and 290 can be referred to as Rogowsky roll-off, providing a reduction in an amount of harmonic error in an entry and exit magnetic dipole field, and reducing pole saturation locally.

A cross-sectional view (e.g., cross-section 2C in FIG. 2A) of an example non-gradient portion 250 of an example dipole magnet 2000 is depicted in FIG. 2C. With reference to FIG. 2C, beam 205 passes within the non-gradient portion 250 formed by the non-gradient top pole face 210NG-1 and non-gradient bottom pole face 210NG-2. For the non-gradient portion 250, respective height dimensions throughout the non-gradient portion can be constant. For example, a height dimension in a middle (not shown in FIG. 2C) of the non-gradient portion 250 can be the same as a left-most non-gradient height dimension and a right-most non-gradient height dimension, which is the distance between pole faces 210NG-1 and 210NG-2.

As the beam 205 passes through the non-gradient portion 250, a direction of travel of the beam 205 may be controlled by applying a magnetic field between the non-gradient top pole face 210NG-1 and non-gradient bottom pole face 210NG-2. The magnetic field may be applied by applying a current to the pair of windings 215 (e.g., 215-1, 215-2). A direction of the applied current (e.g., current applied to the pair of windings 215-1, 215-2) controls the direction of travel of the beam 205. For example, a current applied in a counter-clockwise direction throughout the pair of windings 215-1, 215-2 can cause the beam 205 to travel to the left relative to a vertical central line 240NG (e.g., vertical central line 240A bisects a width of the top pole face 210NG-1 and bottom pole face 210NG-2 of the non-gradient portion 250 of dipole magnet 2000). As another example, a current applied in a clockwise direction throughout the pair of windings 215-1, 215-2 can cause the beam 205 to travel to the right relative to the vertical central line 240NG. Moreover, a strength of the applied magnetic field between the non-gradient top pole face 210NG-1 and non-gradient bottom pole face 210NG-2 may influence a magnitude of bending experienced by the beam 205. For example, a relatively stronger applied magnetic field induced by a relatively larger counter-clockwise current flow (e.g., applied to the pair of windings 215-1, 215-2) can cause the beam 205 to bend further to the left of the vertical central line 240NG as compared to a relatively weaker magnetic field induced by a relatively smaller counter-clockwise current flow (e.g., applied to the pair of windings 215-1, 215-2). In some embodiments, the beam may traverse through the non-gradient portion 250 along a horizontal plane 245NG, which can be defined by a horizontal plane equidistantly positioned between the non-gradient top pole face 210NG-1 and non-gradient bottom pole face 210NG-2.

With reference again to FIG. 2A, the beam 205 may continue to pass through the non-gradient portion 250 and reach a gradient portion 260 formed by gradient portions of the top pole face 210-1 and bottom pole face 210-2 of the dipole magnet 2000. The gradient portion 260 may be formed by a gradient top pole face 210G-1A and 210G-1B (not shown in FIGS. 2A or 2B) and a gradient bottom pole face 210G-2A and 210G-2B (not shown in FIGS. 2A or 2B). Gradient top pole face 210G-1A and 210G-1B and gradient bottom pole face 210G-2A and 210G-2B can each be angled relative to the non-gradient portion 250 and to one another. In some embodiments, a transition or central portion 202 (e.g., shown in FIG. 2B; e.g., a transition from the non-gradient portion 250 to the gradient portion 260) of the dipole magnet 2000 can be flat, substantially flat, or of a particular angle relative to the non-gradient portion 250 so as to provide a soft transition from the non-gradient portion 250 to the gradient portion 260. In various embodiments, there is a separation between a first plane 260A (e.g., having surfaces 210G-1A and 210G-2A) and a second plane 260B (e.g., having surfaces 210G-1B and 210G-2B) where the gradient switches direction. That is, in some embodiments, the gradient portion 260 includes a first plane 260A (e.g., having surfaces 210G-1A and 210G-2A) and a second plane 260B (e.g., having surfaces 210G-1B and 210G-2B). At the end of the top pole face 210-1 and bottom pole face 210-2, the two planes 260A (e.g., having surfaces 210G-1A and 210G-2A) and 260B (e.g., having surfaces 210G-1B and 210G-2B) are at or near a final angle of the bend of the magnet 2000. The separation (e.g., a void) between the first plane 260A (e.g., having surfaces 210G-1A and 210G-2A) and the second plane 260B (e.g., having surfaces 210G-1B and 210G-2B) permits avoidance of undesired or undesirable harmonics for a beam dump (e.g., a straight beam exit) located between two of the beam travel paths provided by the dipole magnet 2000.

As shown in FIGS. 2A and 2B, planes 260A (e.g., having surfaces 210G-1A and 210G-2A) and 260B (e.g., having surfaces 210G-1B and 210G-2B) are at 45 degree angles relative to a center of the dipole magnet 2000 at the exit of the beam. In other embodiments, planes 260A (e.g., having surfaces 210G-1A and 210G-2A) and 260B (e.g., having surfaces 210G-1B and 210G-2B) have different exit angles. For example, in another embodiment, plane 260A (e.g., having surfaces 210G-1A and 210G-2A) is at 45 degrees (e.g., the beam exits at 45 degrees relative to a center of the dipole magnet 2000) while plane 260B (e.g., having surfaces 210G-1B and 210G-2B) is at 60 degrees (e.g., the beam exits at 60 degrees relative to the center of the dipole magnet 2000).

A cross-sectional view (e.g., 2D of FIG. 2A) of an example gradient portion 260 of an example dipole magnet 2000 is depicted in FIG. 2D. With reference to FIG. 2D, the beam 205 can pass through the gradient portion 260 formed by the gradient top pole face 210G-1A and 210G-1B and the gradient bottom pole face 210G-2A and 210G-2B. It will be appreciated that, while FIG. 2D depicts multiple beams 205 for illustrative purposes, in various embodiments, it is possible for only one beam 205 to traverse the dipole magnet 2000 at a time. That is, a beam 205 may travel along a path associated with a gradient portion of plane 260A (e.g., having surfaces 210G-1A and 210G-2A), or the beam 205 may travel along a path associated with a gradient portion of plane 260B (e.g., having surfaces 210G-1B and 210G-2B). A position of the beam 205 within the gradient portion 260 of the dipole magnet 2000 can be based on or impacted by, at least in part, the applied magnetic field (not shown in FIG. 2D) during the traversal of the beam through the non-gradient portion 250 (not shown in FIG. 2D).

In example embodiments, respective height dimensions 220E, 220F, 220G, and/or 220H throughout the gradient portion 260 can be non-constant and unique as compared to any of the other height dimensions. For example, the gradient height dimension 220E can differ from the gradient height dimension 220F. Similarly, the gradient height dimension 220G can differ from the gradient height dimension 220H. In some embodiments, the gradient portion 260 may be symmetrically defined about a vertical central line 240G (e.g., vertical central line 240G bisects a width of the top pole face 210G-1A, 210G-1B and bottom pole face 210G-2A, 210G-2B of gradient portion 260 of dipole magnet 2000), and be configured such that a left-most gradient portion or plane 260A (e.g., having surfaces 210G-1A and 210G-2A) is a substantially mirrored version of the right-most gradient portion or plane 260B (e.g., having surfaces 210G-1B and 210G-2B) and vice versa. As such, the gradient height dimension 220E can be the same as or substantially similar to the gradient height dimension 220H; similarly, the gradient height dimension 220F can be the same as or substantially similar to the gradient height dimension 220G.

In some embodiments, the beam 205 can pass through the gradient portion 260 along a horizontal plane 245G (e.g., horizontal plane 245 can be defined by a horizontal plane equidistantly positioned between the gradient top pole face 210G-1A and 210G-1B and gradient bottom pole face 210G-2A and 210G-2B). As the beam 205 passes through the gradient portion 260 of the dipole magnet 2000, the beam 205 may be focused in both transverse directions. As such, the dipole magnet 2000 may focus the beam 205 and thus eliminate the need for the inclusion of a quadrupole magnet in the beamline of an example beam system 10.

A cross-sectional view (e.g., 2E of FIG. 2A) of an example gradient portion 260 of an example dipole magnet 2000 is depicted in FIG. 2E. In FIG. 2E, the beam 205 can pass through a gradient portion 260 along a horizontal plane 245G and to the right of a vertical central line 240G bisecting a width of the top pole face 210G-1A and 210G-1B and bottom pole face 210G-2A and 210G-2B of gradient portion 260 of dipole magnet 2000.

A cross-sectional view (e.g., 2F of FIG. 2A) of an example gradient portion 260 of an example dipole magnet 2000 is depicted in FIG. 2F. In FIG. 2F, the beam 205 can pass through a gradient portion 260 along a horizontal plane 245G and to the left of a vertical central line 240G bisecting a width of the top pole face 210G-1A and 210G-1B and bottom pole face 210G-2A and 210G-2B of gradient portion 260 of dipole magnet 2000.

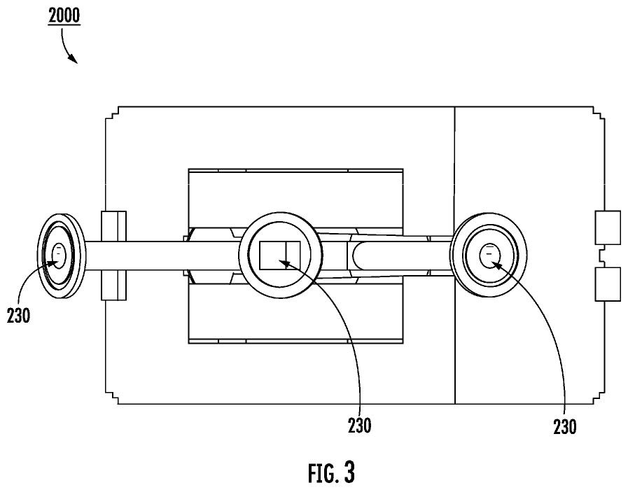

As shown in FIG. 3, an example beam system 10 can include one or more beam tubes 230 configured to inject a beam (not shown in FIG. 3) into example dipole magnet 2000 and permit the dipole magnet 2000 to adjust a beam propagation direction and focal characteristics. In some embodiments, the one or more beam tubes 230 can be vertically and/or horizontally positioned in between a top pole face (not shown in FIG. 3) and bottom pole face (not shown in FIG. 3) of the dipole magnet 2000. As an example, the beam tubes 230 can be attached to a single vacuum weldment occupying most of the volume between the pole faces contained within 210NG-1, 210NG-2, 210G-1A and 210G-1B, 210G-2A and 210G-2B in FIGS. 2C, 2D, 2E, and 2F.

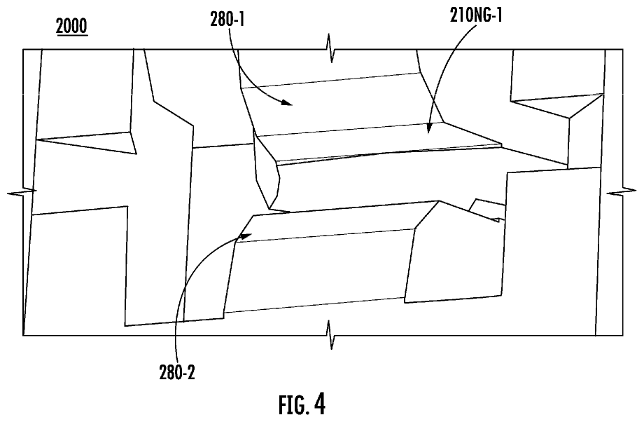

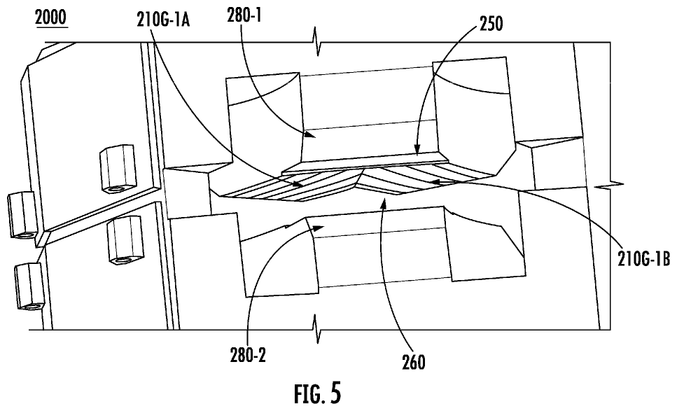

Turning to FIG. 4, an example injection entryway for an example dipole magnet 2000 of example beam system 10 is shown. In some embodiments, prior to the non-gradient portion 250 of the dipole magnet 2000, chamfered edges 280-1 and 280-2 can be included on the top pole face 210NG-1 and bottom pole face 210NG-2 (not shown in FIG. 4), respectively, and extend along an edge that is perpendicular to a direction of travel of an injected beam. That is, chamfered edges 280-1 and 280-2 can create a gradient in the direction of travel of the injected beam, but the chamfered edges 280-1 and 280-2 do not create a gradient transverse to the direction of travel of the injected beam. As mentioned above, chamfered edges 280-1, 280-2 can be referred to as Rogowsky roll-off, providing a reduction in an amount of harmonic error in an entry and exit magnetic dipole field, and reducing pole saturation locally.

In some embodiments, each chamfered edge 280-1 and 280-2 can form an angle (e.g., approximately +/−45°) relative to horizontal plane 245NG (not shown in FIG. 4). For example, the chamfered edge 280-1 included on the top pole face 210NG-1 can be associated with a first angle (e.g., approximately +45°) with respect to the horizontal plane 245NG while the chamfered edge 280-2 included on the bottom pole face 210NG-2 (not shown) can be associated with a second angle (e.g., approximately −45°) with respect to the horizontal plane 245NG. It will be appreciated that a threshold error or range (e.g., up to +/−5°) with respect to the angles of the chamfered edges relative to the horizontal planes can be tolerated in various embodiments.

The chamfered edges 280-1 and 280-2 (e.g., and/or 290) can substantially reduce or eliminate undesirable magnetic field end harmonics that can potentially damage or perturb the profile of an injected beam. In some embodiments, a chamfered edge (e.g., 280-1, 280-2) near the injection entryway can be used to bend, or otherwise aid the non-gradient portion 250 of the bidirectional dipole magnet 2000 in bending, an injected beam 205 in one of multiple possible directions (e.g., left or right relative to a center or central line of a beamline, or other directions).

In some embodiments, a uniform magnetic field (e.g., or substantially uniform magnetic field) is generated in between the non-gradient top pole face 210NG-1 and non-gradient bottom pole face 210NG-2 of the non-gradient portion 250 of the dipole magnet 2000. In some embodiments, the uniform magnetic field generated between the non-gradient top pole face 210NG-1 and non-gradient bottom pole face 210NG-2 of the non-gradient portion 250 of the dipole magnet 2000 is configured to bend an injected beam 205 in at least two (e.g., two or more) possible directions along two (e.g., two or more) corresponding beam paths. In some embodiments, the at least two directions can be angled (e.g., approximately +45 and −45 degrees) relative to a vertical central line (e.g., 240NG, 240G). In some embodiments, the at least two directions can further include −90, −45, 0, +45, and +90 degrees relative to the vertical central line 240 (e.g., 240NG, 240G). It will be appreciated that any number of path directions or angles of path directions for a beam path can be within the scope of the present disclosure.

A direction of the magnetic field produced by the dipole magnet 2000 can at least in part control the bending of the beam. A strength of the magnetic field produced by the dipole magnet 2000 can also control the magnitude of bending of the beam. The non-gradient portion 250 of the dipole magnet 2000 can extend toward or into each desired beam path or direction until a beam passing therethrough would no longer be impacted by effects of the magnetic field produced by the other beam path or direction upon arriving at the gradient portion of the dipole magnet 2000.

Turning to FIG. 5, an example dipole magnet 2000 is shown to include gradient portion 260 as formed by a gradient top pole face 210G-1A and 210G-1B and gradient bottom pole face 210G-2A and 210G-2B (not shown in FIG. 5). In some embodiments, the gradient portion 260 is configured with a slope (e.g., between 5-15 degrees) angled away from a horizontal central line 245G (not shown in FIG. 5), which bisects a height between top pole face (not shown in FIG. 5) and bottom pole face (not shown in FIG. 5) of the dipole magnet 2000. As such, the magnetic field produced between the gradient top pole face 210G-1A and 210G-1B and gradient bottom pole face 210G-2A and 210G-2B of the dipole magnet 2000 is non-uniform. This non-uniform magnetic field of the gradient portion 260 of the dipole magnet 2000 can focus the beam in both transverse directions. In some embodiments, the gradient or slope (n) between top gradient pole face 210G-1A and 210G-1B and bottom gradient pole face 210G-2A and 210G-2B is 0.5 or 0.15. As such, the beam system 10 can both bend the beam (e.g., to one of multiple directions) as well as focus the beam using the dipole magnet 2000. This can eliminate the need for a quadrupole magnet downstream along the beamline, resulting in a shorter beamline and less complex focusing scheme. Eliminating the need for a quadrupole magnet downstream along the beamline permits direct travel from the dipole magnet (e.g., 2000) to a raster without an additional intervening optical component between the dipole and the raster.

Moreover, in addition to a more compact beamline, as a result of the inclusion of the present dipole magnet (e.g., 2000), eliminating components from the beamline results in fewer components that can become radioactive and therefore difficult to maintain. Fewer components in a beamline results in shorter distance that a beam must travel through the beamline to the target, and a shorter distance leads to smaller magnitudes of fluctuations in beam parameters that need to be adjusted or controlled. Beamline component parameters can be easily adjusted in near real-time when the fluctuations are of smaller magnitudes.

In some embodiments, the transition 202 between the non-gradient portion 250 to the gradient pole face portion 260 is sharp such that the gradient portion 260 of the dipole magnet 2000 immediately transitions to the desired slope of the non-gradient portion 250. For example, the gradient portion 260 of dipole magnet 2000 can immediately transition to a slope of +15 for the gradient top pole face 210G-1A and 210G-1B and −15 degrees for the gradient bottom pole face 210G-2A and 210G-2B. In some embodiments, the transition 202 between the non-gradient portion 250 to the gradient portion 260 is gradual such that the gradient pole face portion 260 of the dipole magnet 2000 gradually transitions to the desired slope. For example, the gradient pole face portion 260 of dipole magnet 2000 can begin with a slope of +5 for the gradient top pole face and −5 degrees for the gradient bottom pole face and increase up to a slope of +15 and −15 degrees, respectively.

Dimensions and parameters of components of the dipole magnets herein are configured to permit control of a beam traveling through a dipole magnet such that the beam traveling through the dipole magnet is directed along one of multiple desired beam travel paths without being undesirably impacted by the existence of the other possible beam travel paths. For example, the non-gradient portion 250 preferably has a distance or length that provides for a magnetic field within the non-gradient portion 250 to influence a direction of travel of a beam passing therethrough so that, when the beam passing therethrough arrives at the gradient portion 260, a magnetic field within the gradient portion 260 can further influence a direction of travel of the beam along a desired beam travel path without the beam being undesirably impacted by the magnetic field within the gradient portion 260 into traveling along a different beam travel path of the dipole magnet 2000.

Once the distance between the non-gradient portion 250 of the dipole magnet 2000 is sufficiently long such that an injected beam is no longer impacted by effects of the magnetic field associated with a beam direction other than the desired beam direction, the dipole magnet 2000 can transition from a non-gradient portion 250 to a gradient portion 260. In some embodiments, the transition to a gradient portion 260 can occur once a beam is deflected over three times the size of its associated beam diameter. As such, this prevents the beam from being perturbed by the magnetic field produced by the gradient portion 260 of the dipole magnet 2000 corresponding to one of the other beam paths.

In some embodiments, when a beam is injected, the injected beam (e.g., 205) can pass between the non-gradient top pole face 210NG-1 and non-gradient bottom pole face 210NG-2. A vertical central line 240NG may bisect the width of the non-gradient top pole face 210NG-1 and non-gradient bottom pole face 210NG-2. A magnetic field produced between the non-gradient top pole face 210NG-1 and non-gradient bottom pole face 210NG-2, by way of applying current to windings 215, can direct the beam to the left or right of the vertical central line 240NG. The non-gradient face portion 250 of the dipole magnet 2000 may diverge into two or more beam paths before transitioning to the gradient portion 260 (e.g., including gradient top pole face 210G-1A and 210G-1B and gradient bottom pole face 210G-2A and 210G-2B) of the dipole magnet 2000.

Embodiments herein provide for a more compact beamline that is able to provide multiple paths into one of many treatment rooms. That is, for example, two or more beam paths may provide a path into a particular room in a facility, such as a treatment room in a hospital facility. The beam may be directed to a particular beam path of the two or more beam paths using the dipole magnet (e.g., 2000) and into a room (e.g., or target) within the facility. For example, a room A and a room B may be adjacent to one another and a first beam path of the beam system (e.g., 10) may lead to room A while a second beam path of the beam system (e.g., 10) may lead to room B. If a patient is in room B awaiting treatment that involves the beam (e.g., 205), the beam (e.g., 205) may be efficiently and safely directed to the second beam path using the methods described herein. Additionally, when the beam (e.g., 205) arrives at the patient in room B, embodiments herein ensure the beam (e.g., 205) is properly focused and of the desired beam diameter (e.g., or other parameters).

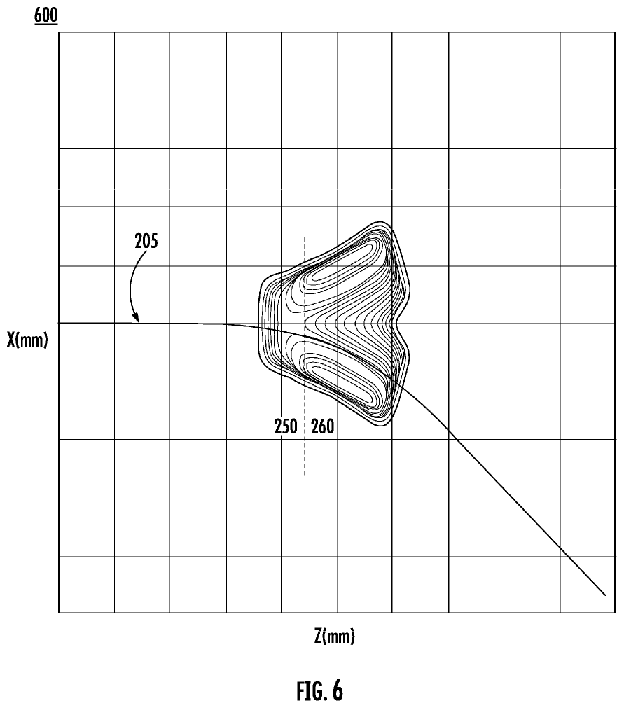

FIG. 6 depicts an example magnetic field distribution 600 at the midplane of a beam system 10. A beam 205 can be injected into a magnetic field produced between a top pole face and bottom pole face of a dipole magnet 2000 of example embodiments herein. Here, the magnetic field ranges from 0.40 tesla (T) to 0.48 T. The outermost, exterior portions of the magnetic field distribution can be the result of end harmonics of the magnetic field produced between top pole face and bottom pole face of the dipole magnet 2000. The magnetic field can be seen to be mostly uniform for the non-gradient portion 250. This is due to the uniform magnetic field produced by non-gradient top pole face 210NG-1 and non-gradient bottom pole face 210NG-2 of the bidirectional dipole magnet 2000. For the gradient portion 260, the magnetic field lines can be seen with a gradient in at least two directions. This is due to the non-uniform magnetic field produced by gradient top pole face 210G-1A and 210G-1B and gradient bottom pole face 210G-2A and 210G-2B. For example, the gradient portion 260 can be seen to include magnetic field lines indicative of magnetic field strengths between 0.40 T to 0.48 T.

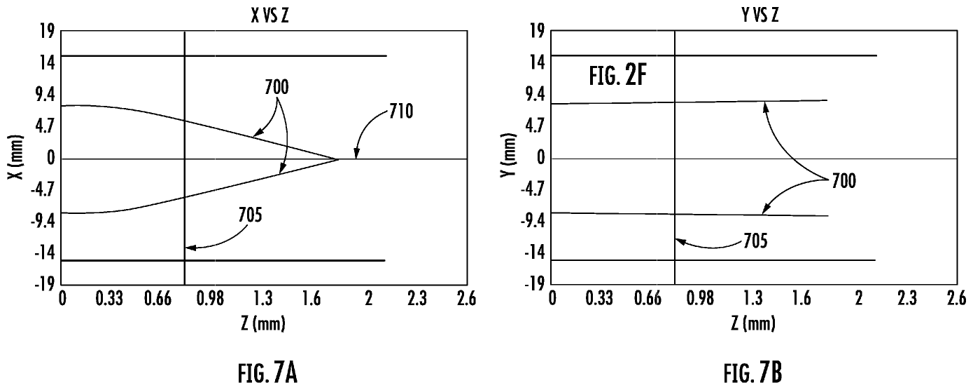

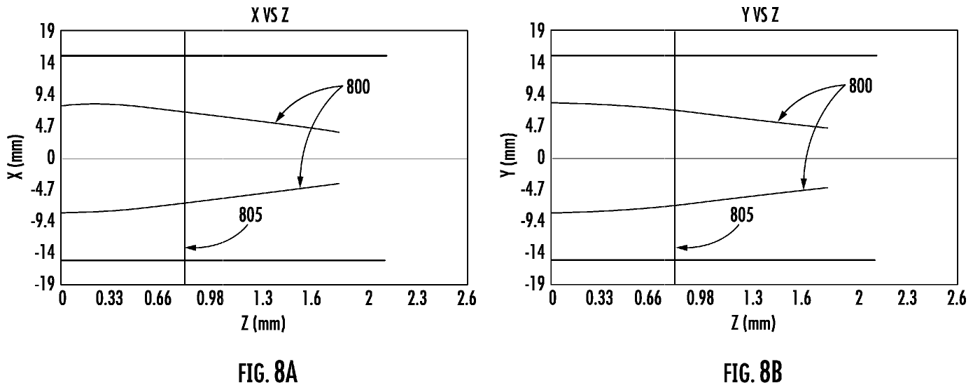

Turning now to FIGS. 7A-7B, an example profile of a beam envelope 700 in both transverse directions is shown for a beam traveling (e.g., in parallel) along a beam path having only a non-gradient pole face portion of a bidirectional dipole magnet. In the example depicted in FIGS. 7A and 7B, the length of the bidirectional dipole magnet extends from 0 m to 0.8 m along the z-axis, where 0.8 m is represented by the vertical line 705. As shown in FIG. 7A, because the bidirectional dipole magnet only includes a non-gradient portion (e.g., as opposed to both a non-gradient portion as well as a gradient portion in embodiments herein), the beam can become over-focused in one transverse direction (e.g., the x axis) such that the beam collapses into a single point 710. Not only can this result in beam collapse as is shown in FIG. 7A, but also a loss of the beam and possible damage to beamline components. In such examples, a quadrupole magnet may be required prior to the dipole magnet to compensate for this over focusing. Alternatively, as shown in FIG. 7B, bidirectional dipole magnets which only include a non-gradient portion (e.g., as opposed to both a non-gradient portion as well as a gradient portion in embodiments herein) are unable to perform any focusing in the other transverse direction (e.g., the y axis). This lack of beam focusing then requires an additional quadrupole magnet further down the beamline to properly focus the beam to a desired size at a target.

FIGS. 8A-8B show an example profile of a beam envelope 800 for a beam within a bidirectional dipole magnet which includes a gradient portion. As shown in FIGS. 8A-8B, the beam profile can be focused in both transverse directions. Shown in FIGS. 8A and 8B, the length of the bidirectional dipole magnet extends along the z axis from 0 m to 0.8 m, where 0.8 m is indicated by vertical line 805. As shown in FIG. 8A, because the dipole magnet (e.g., 2000) includes a gradient portion, the beam can become focused but does not become overly-focused in one transverse direction (e.g., the x axis) relative to the other transverse direction (e.g., the y axis). Similarly, as seen in FIG. 8B, because the bidirectional dipole magnet includes a gradient portion, the bidirectional dipole magnet can focus the beam in the other transverse direction (e.g., the y axis) as well. That is, inclusion of the gradient portion within the bidirectional dipole magnet permits the ability to focus the beam in both transverse directions without overfocusing the beam in one transverse direction relative to the other.

BNCT based on tandem accelerators generally have a small footprint, but size and safety constraints continue to present drawbacks associated with buildings or spaces that are unable to accommodate the systems. In particular, it is difficult to shield critical accelerator components that have direct line of sight to the lithium target.

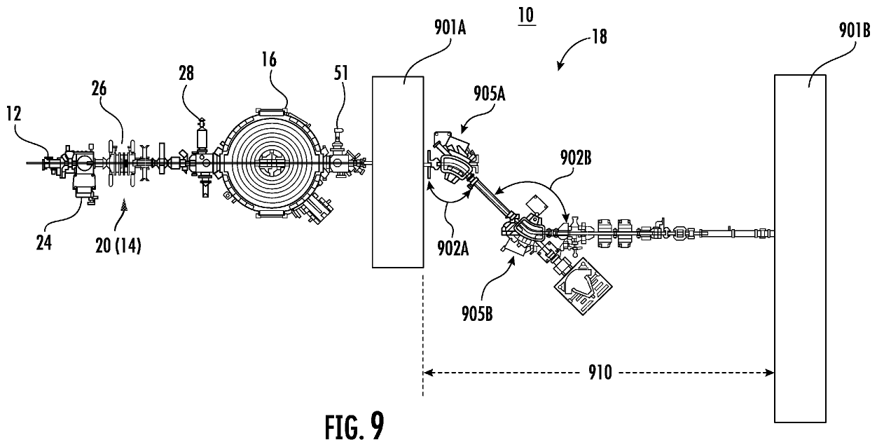

FIG. 9 illustrates an example beam system usable with embodiments of the present disclosure. In FIG. 9, an example beam system 10 includes an ion source 12, a pre-accelerator system 20 (e.g., or 14), an accelerator 16, and an HEBL 18. A first neutronics shielding wall 901A is positioned downstream of accelerator 16, and a second neutronics shielding wall 901B is positioned downstream of HEBL 18 prior to or upstream of a treatment room (not shown). One or more first magnets 905A (e.g., quadrupole magnets 52, 72, and/or dipole or bending magnets 56, 58, 2000), positioned downstream from the first neutronics shielding wall 901A, change the trajectory of the particle beam and permit a first bend in HEBL 18 having an angle 902A. Angle 902A can be 45°+/−5°, or other angles as appropriate for the specific implementation. One or more second magnets 905B (e.g., quadrupole magnets 52, 72, and/or dipole or bending magnets 56, 58, 2000), positioned downstream from the one or more first magnets 905A and upstream from the second neutronics shielding wall 901B, permit a second bend in HEBL 18 having an angle 902B. In some embodiments, angle 902A and angle 902B each can be 45°+/−5°, or other angles as appropriate for the specific implementation. A distance 910 between the first neutronics shielding wall 901A and the second neutronics shielding wall 901B can be minimized. The first neutronics shielding wall 901A can be positioned parallel to the second neutronics shielding wall 901B. In embodiments in accordance with FIG. 9, a larger beam size may result at the one or more first magnets 905A (e.g., downstream from the accelerator 16). Such larger beam size may be mitigated by placing a quadrupole magnet pair (not shown) in the first neutronics shielding wall 901A. The first neutronics shielding wall 901A can also include a radiation shielding complexity associated with a larger and more complex wall opening (e.g., for passage of the beam).

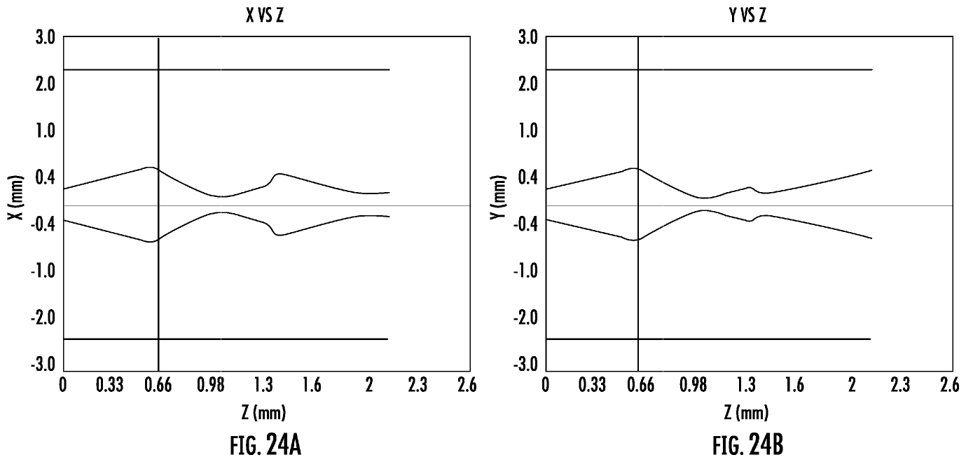

FIGS. 24A-24B show an example profile of a beam envelope for a beam within a beam system using a pair of quadrupole magnets, providing for respective angles of 45°, where the quadrupole magnets are positioned downstream of a neutronics shielding wall that is between an accelerator and the quadrupole magnets (e.g., in example embodiments shown in FIG. 9).

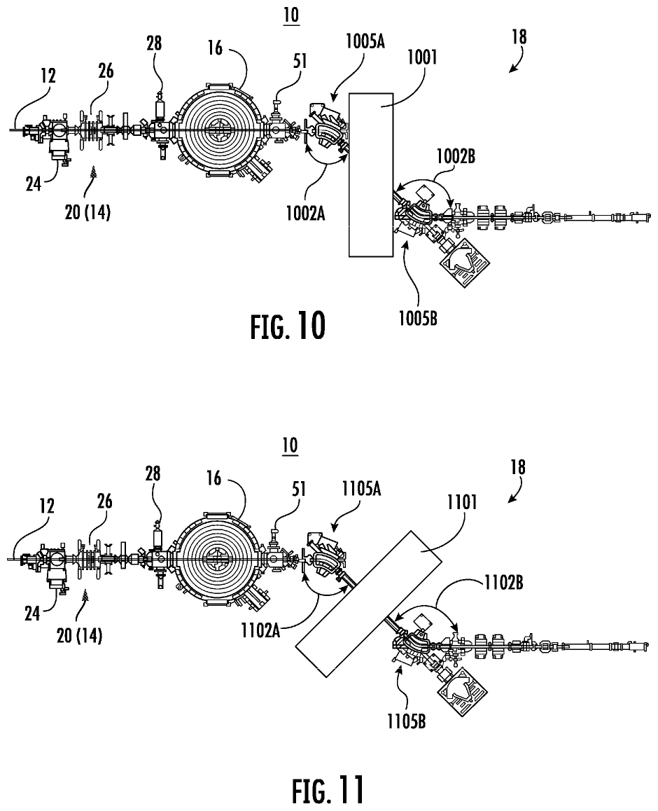

FIG. 10 illustrates an example beam system usable with embodiments of the present disclosure. In FIG. 10, an example beam system 10 includes an ion source 12, a pre-accelerator system 20 (e.g., or 14), an accelerator 16, and an HEBL 18. A neutronics shielding wall 1001 is positioned downstream of accelerator 16 one or more first magnets 1005A positioned downstream of the accelerator 16. The one or more first magnets 1005A (e.g., quadrupole magnets 52, 72, and/or dipole or bending magnets 56, 58, 2000) permit a first bend in the beam line having an angle 1002A. One or more second magnets 1005B (e.g., quadrupole magnets 52, 72, and/or dipole or bending magnets 56, 58, 2000), positioned downstream from the neutronics shielding wall 1001, permit a second bend in the beam line having an angle 1002B. In some embodiments, angle 1002A and angle 1002B each can be 45°+/−5°, or other angles as appropriate for the specific implementation. The neutronics shielding wall 1001 can be positioned parallel to the exit of the accelerator 16 and therefore a beam passing therethrough passes at an angle (e.g., 45°+/−5°, or other angles as appropriate for the specific implementation) relative to the accelerator 16 exit.

FIG. 11 illustrates an example beam system usable with embodiments of the present disclosure. In FIG. 11, an example beam system 10 includes an ion source 12, a pre-accelerator system 20 (e.g., or 14), an accelerator 16, and an HEBL 18. A neutronics shielding wall 1101 is positioned downstream of accelerator 16 one or more first magnets 1105A positioned downstream of the accelerator 16. The one or more first magnets 1105A (e.g., quadrupole magnets 52, 72, and/or dipole or bending magnets 56, 58, 2000) permit a first bend in the beam line having an angle 1102A. One or more second magnets 1105B (e.g., quadrupole magnets 52, 72, and/or dipole or bending magnets 56, 58, 2000), positioned downstream from the neutronics shielding wall 1101, permit a second bend in the beam line having an angle 1102B. In some embodiments, angle 1002A and angle 1002B each can be 45°+/−5°, or other angles as appropriate for the specific implementation. The neutronics shielding wall 1101 can be positioned at an angle (e.g., 45°+/−5°, or other angles as appropriate for the specific implementation) relative to the exit of the accelerator 16 and therefore a beam passing therethrough passes perpendicular to the neutronics shielding wall 1101.

The example embodiments depicted in FIGS. 10 and 11 may employ a first dipole magnet (e.g., 2000) as the one or more first magnets (e.g., 1005A or 1105A) and a second dipole magnet (e.g., 2000) as the one or more second magnets (e.g., 1005B or 1105B). In example embodiments, the first dipole magnet and the second dipole magnet can be separated with an approximately two meter drift section. Such a configuration permits multiple shielding options (e.g., the shielding wall configuration of FIG. 10 or the shielding wall configuration of FIG. 11). Additionally or alternatively, a quadrupole magnet (not shown) can be used as well to compensate for accelerator-related horizontal over-focus.

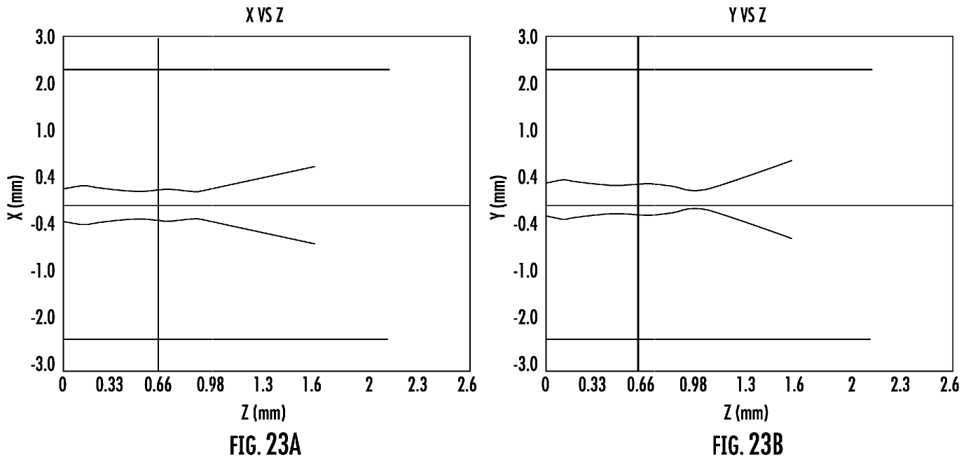

FIGS. 23A-23B show an example profile of a beam envelope for a beam within a beam system using a pair of dipole magnets (e.g., 2000) providing for respective angles of 45° (e.g., in example embodiments shown in FIGS. 10-11).

In embodiments, dipole magnets (e.g., 2000) can be used in place of quadrupole magnets, which can lead to a more compact system design because a single dipole magnet can provide the functionality (or at least some of the functionality) of two quadrupole magnets. A single dipole magnet leads to a smaller overall footprint than a system design having two quadrupole magnets in the same position. Accordingly, a system design employing dipole magnets where a pair of quadrupole magnets would otherwise have been used will have a smaller overall footprint. A smaller footprint, or a more compact system design, permits the use of the system in smaller spaces or space-constrained locations.

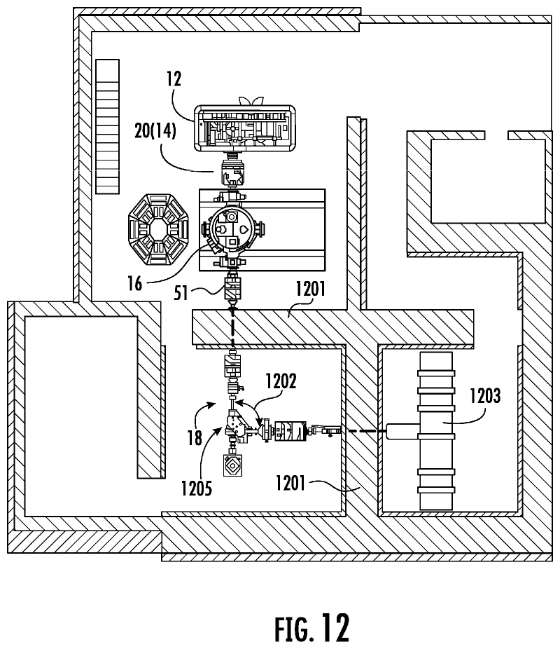

FIG. 12 illustrates an example beam system usable with embodiments of the present disclosure. In FIG. 12, an example beam system includes an ion source 12, a pre-accelerator system 20 (e.g., or 14), an accelerator 16, and an HEBL 18. A treatment room 1203 is positioned downstream of accelerator 16. One or more magnets 1205 (e.g., quadrupole magnets 52, 72, and/or dipole or bending magnets 56, 58, 2000) are positioned downstream of accelerator 16, providing for a beam passing through the system 10 to be steered toward treatment room 1203 at an angle 1202. A neutronics shielding wall 1201 is positioned downstream of accelerator 16 and upstream of the one or more magnets 1205, as well as downstream of the one or more magnets and the treatment room 1203. In some embodiments, angle 1202 can be 90°+/−5°, or other angles as appropriate for the specific implementation. While the embodiment depicted in FIG. 12 shows neutronics shielding wall 1201 to be continuous (e.g., a first portion of neutronics shielding wall positioned downstream of accelerator 16 as well as a second portion of neutronics shielding wall positioned upstream of treatment room 1203), it will be appreciated that neutronics shielding wall 1201 can include multiple separate portions.

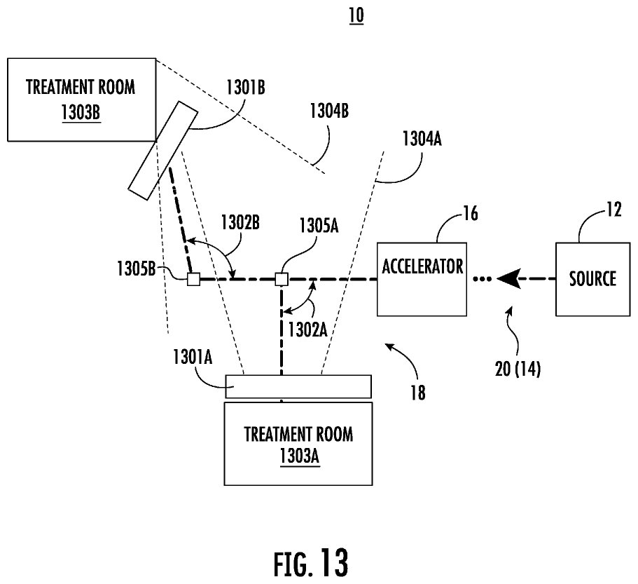

FIG. 13 illustrates an example beam system usable with embodiments of the present disclosure. In FIG. 13, an example beam system 10 includes an ion source 12, a pre-accelerator system 20 (e.g., or 14), an accelerator 16, and an HEBL 18. Treatment rooms 1303A and 1303B are positioned downstream from accelerator 16 and HEBL 18, and are situated on opposite sides of a beamline relative to one another. One or more first magnets 1305A (e.g., quadrupole magnets 52, 72, and/or dipole or bending magnets 56, 58, 2000) are positioned along the beam line such that a beam passing through the beam line intended for treatment room 1303A is steered at an angle 1302A toward treatment room 1303A. A first neutronics shielding wall 1301A is positioned between the one or more first magnets 1305A and the treatment room 1303A. As a result of the beam encountering first neutronics shielding wall 1301A and/or treatment room 1303A, neutrons can travel away from first treatment room 1303A and/or first neutronics shielding wall 1301A according to a first neutron back-travel zone 1304A depicted in dashed lines.

One or more second magnets 1305B (e.g., quadrupole magnets 52, 72, and/or dipole or bending magnets 56, 58, 2000) are positioned along the beam line, downstream from the first one or more magnets 1305A, such that a beam passing through the beam line intended for treatment room 1303B is steered toward treatment room 1303B at an angle 1302B. A second neutronics shielding wall 1301B is positioned between the one or more second magnets 1305B and treatment room 1303B. As a result of the beam encountering second neutronics shielding wall 1301B and/or treatment room 1303B, neutrons can travel away from treatment room 1303B and/or second neutronics shielding wall 1301B according to a second neutron back-travel zone 1304B depicted in dashed lines.

In embodiments, angle 1302A can be 90°+/−5°, or other angles as appropriate for the specific implementation and angle 1302B can be 120°+/−5°, or other angles as appropriate for the specific implementation. The first neutronics shielding wall 1301A can be positioned such that a beam passing therethrough passes perpendicular to the first neutronics shielding wall 1301A.

Distances, dimensions, layout, and angles associated with first treatment room 1303A, first neutronics shielding wall 1301A, as well as second treatment room 1303B and second neutronics shielding wall 1301B are configured such that components of system 10 are protected from the neutron back-travel zones 1304A and 1304B. For example, the second neutronics shielding wall 1301B can be positioned such that a beam passing therethrough passes at an angle relative to the second neutronics shielding wall 1301B, where the angle ensures the neutron back-travel zone 1304B is angled safely to avoid damage to system 10 components (e.g., travel of neutrons within zone 1304B is not directed toward equipment).

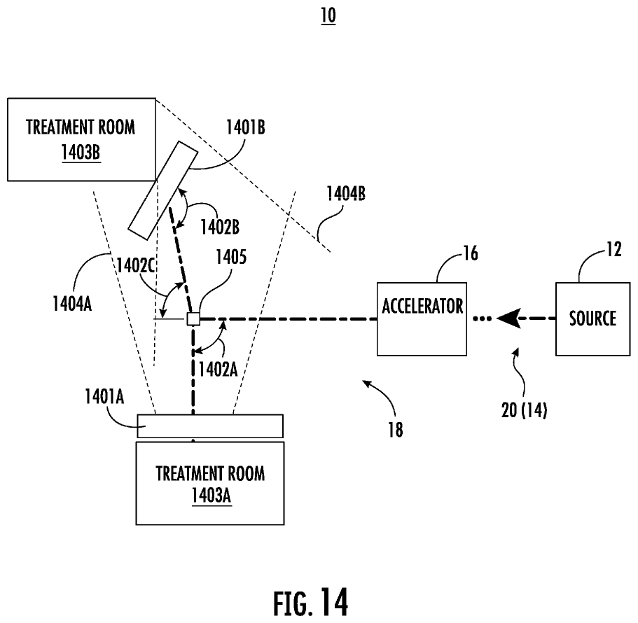

FIG. 14 illustrates an example beam system usable with embodiments of the present disclosure. In FIG. 14, an example beam system 10 includes an ion source 12, a pre-accelerator system 20 (e.g., or 14), an accelerator 16, and an HEBL 18. Treatment rooms 1403A and 1403B are positioned downstream from accelerator 16 and HEBL 18, and are situated on opposite sides of a beamline relative to one another. A magnet 1405 (e.g., dipole magnet 2000) is positioned along the beam line such that a beam passing through the beam line intended for treatment room 1403A is steered at an angle 1402A toward treatment room 1403A, and such that the beam is also steered at an angle 1402C toward treatment room 1403B. A first neutronics shielding wall 1401A is positioned between the magnet 1405 and the treatment room 1403A. As a result of the beam encountering first neutronics shielding wall 1401A and/or treatment room 1403A, neutrons can travel away from first treatment room 1403A and/or first neutronics shielding wall 1401A according to a first neutron back-travel zone 1404A depicted in dashed lines.

A second neutronics shielding wall 1401B is positioned between the magnet 1405 and treatment room 1403B. As a result of the beam encountering second neutronics shielding wall 1401B and/or treatment room 1403B, neutrons can travel away from treatment room 1403B and/or second neutronics shielding wall 1401B according to a second neutron back-travel zone 1404B depicted in dashed lines.

In embodiments, angle 1402A can be 90°+/−5°, or other angles as appropriate for the specific implementation and angle 1402C can be 60°+/−5°, or other angles as appropriate for the specific implementation. The first neutronics shielding wall 1401A can be positioned such that a beam passing therethrough passes perpendicular to the first neutronics shielding wall 1401A. The second neutronics shielding wall 1401B can be positioned such that the beam passing therethrough passes at an angle 1402B relative to the second neutronics shielding wall 1401B. In some embodiments, angle 1402B can be 120°+/−5°, or other angles as appropriate for the specific implementation. In embodiments, the second neutronics shielding wall 1401B may include a magnet (not shown) to support the angle 1402B and achieve a compact system.

Distances, dimensions, layout, and angles associated with first treatment room 1403A, first neutronics shielding wall 1401A, as well as second treatment room 1403B and second neutronics shielding wall 1401B are configured such that components of system 10 are protected from the neutron back-travel zones 1404A and 1404B.

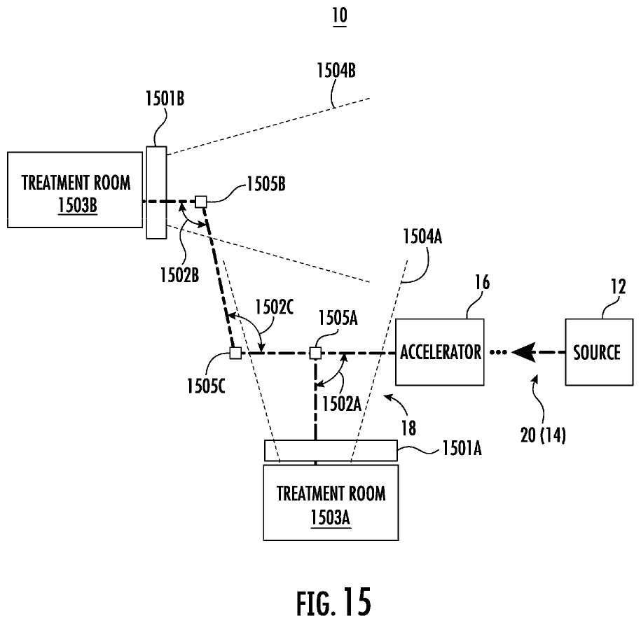

FIG. 15 illustrates an example beam system usable with embodiments of the present disclosure. In FIG. 15, an example beam system 10 includes an ion source 12, a pre-accelerator system 20 (e.g., or 14), an accelerator 16, and an HEBL 18. Treatment rooms 1503A and 1503B are positioned downstream from accelerator 16 and HEBL 18, and are situated on opposite sides of a beamline relative to one another. One or more first magnets 1505A (e.g., quadrupole magnets 52, 72, and/or dipole or bending magnets 56, 58, 2000) are positioned along the beam line such that a beam passing through the beam line intended for treatment room 1503A is steered at a first angle 1502A toward treatment room 1503A. A first neutronics shielding wall 1501A is positioned between the one or more first magnets 1505A and the treatment room 1503A. As a result of the beam encountering first neutronics shielding wall 1501A and/or treatment room 1503A, neutrons can travel away from first treatment room 1503A and/or first neutronics shielding wall 1501A according to a first neutron back-travel zone 1504A depicted in dashed lines.

One or more second magnets 1505C (e.g., quadrupole magnets 52, 72, and/or dipole or bending magnets 56, 58, 2000) are positioned downstream of the one or more first magnets 1505A, and such that the beam is steered at a second angle 1502C toward treatment room 1503B. One or more third magnets 1505B (e.g., quadrupole magnets 52, 72, and/or dipole or bending magnets 56, 58, 2000) are positioned between the one or more second magnets 1505C and a second neutronics shielding wall 1501B such that the beam is steered at a third angle 1502B toward treatment room 1503B.

The second neutronics shielding wall 1501B is positioned between the one or more third magnets 1505B and treatment room 1503B. As a result of the beam encountering second neutronics shielding wall 1501B and/or treatment room 1503B, neutrons can travel away from treatment room 1503B and/or second neutronics shielding wall 1501B according to a second neutron back-travel zone 1504B depicted in dashed lines.

In embodiments, angle 1502A can be 90°+/−5°, or other angles as appropriate for the specific implementation, angle 1502B can be 120°+/−5°, or other angles as appropriate for the specific implementation, and angle 1502C can be 120°+/−5°, or other angles as appropriate for the specific implementation. The first neutronics shielding wall 1501A can be positioned such that a beam passing therethrough passes perpendicular to the first neutronics shielding wall 1501A. The second neutronics shielding wall 1501B can be positioned such that the beam passing therethrough passes perpendicular to the second neutronics shielding wall 1501B.

Distances, dimensions, layout, and angles associated with first treatment room 1503A, first neutronics shielding wall 1501A, as well as second treatment room 1503B and second neutronics shielding wall 1501B are configured such that components of system 10 are protected from the neutron back-travel zones 1504A and 1504B.

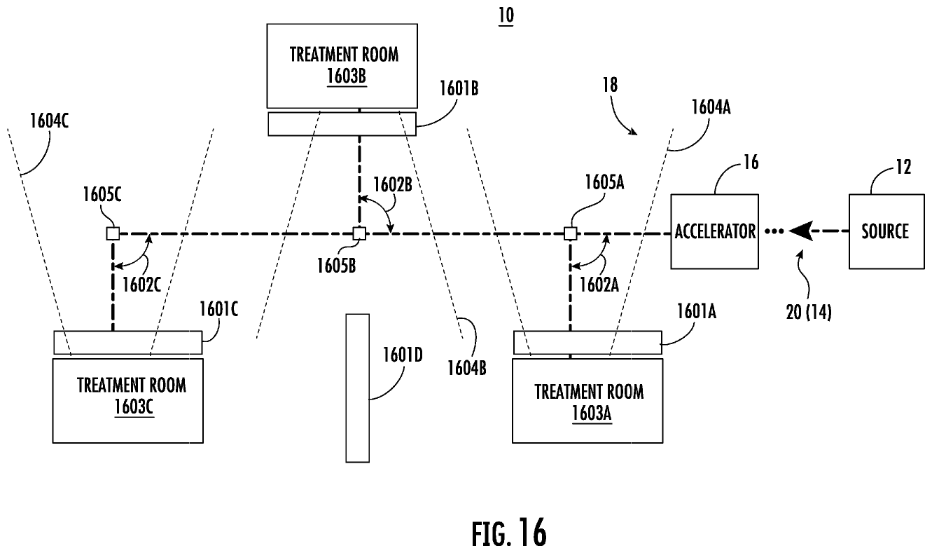

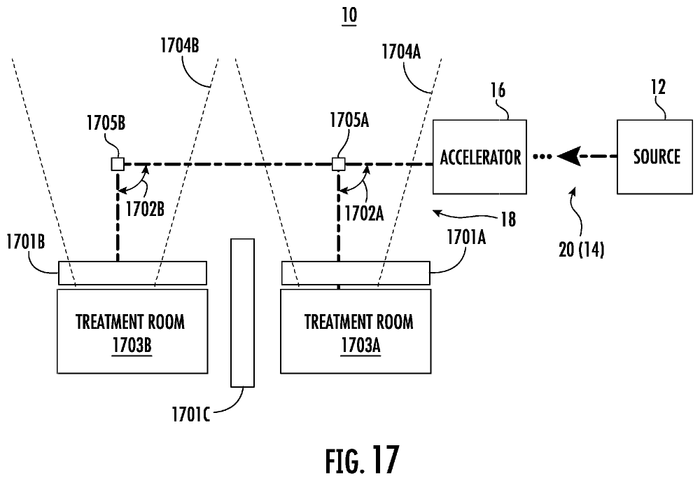

FIG. 16 illustrates an example beam system usable with embodiments of the present disclosure. In FIG. 16, an example beam system 10 includes an ion source 12, a pre-accelerator system 20 (e.g., or 14), an accelerator 16, and an HEBL 18. Treatment rooms 1603A, 1603B, and 1603C are positioned downstream from accelerator 16 and HEBL 18. Treatment rooms 1603A and 1603C are positioned on a same side of the beamline while treatment room 1603B is positioned on an opposite side of the beamline relative to treatment rooms 1603A and 1603C. One or more first magnets 1605A (e.g., quadrupole magnets 52, 72, and/or dipole or bending magnets 56, 58, 2000) are positioned along the beam line such that a beam passing through the beam line intended for treatment room 1603A is steered at a first angle 1602A toward treatment room 1603A. A first neutronics shielding wall 1601A is positioned between the one or more first magnets 1605A and the treatment room 1603A. As a result of the beam encountering first neutronics shielding wall 1601A and/or treatment room 1603A, neutrons can travel away from first treatment room 1603A and/or first neutronics shielding wall 1601A according to a first neutron back-travel zone 1604A depicted in dashed lines.

One or more second magnets 1605B (e.g., quadrupole magnets 52, 72, and/or dipole or bending magnets 56, 58, 2000) are positioned downstream of the one or more first magnets 1605A, and such that the beam is steered at a second angle 1602B toward treatment room 1603B. A second neutronics shielding wall 1601B is positioned between the one or more second magnets 1605B and treatment room 1603B. As a result of the beam encountering second neutronics shielding wall 1601B and/or treatment room 1603B, neutrons can travel away from treatment room 1603B and/or second neutronics shielding wall 1601B according to a second neutron back-travel zone 1604B depicted in dashed lines.

One or more third magnets 1605C (e.g., quadrupole magnets 52, 72, and/or dipole or bending magnets 56, 58, 2000) are positioned downstream of the one or more second magnets 1605B, and such that the beam is steered at a third angle 1602C toward treatment room 1603C. A third neutronics shielding wall 1601C is positioned between the one or more third magnets 1605C and treatment room 1603C. As a result of the beam encountering third neutronics shielding wall 1601C and/or treatment room 1603C, neutrons can travel away from treatment room 1603C and/or third neutronics shielding wall 1601C according to a third neutron back-travel zone 1604C depicted in dashed lines.

In embodiments, angle 1602A can be 90°+/−5°, or other angles as appropriate for the specific implementation, angle 1602B can be 90°+/−5°, or other angles as appropriate for the specific implementation, and angle 1602C can be 90°+/−5°, or other angles as appropriate for the specific implementation. The first neutronics shielding wall 1601A can be positioned such that a beam passing therethrough passes perpendicular to the first neutronics shielding wall 1601A. The second neutronics shielding wall 1601B can be positioned such that the beam passing therethrough passes perpendicular to the second neutronics shielding wall 1601B. The third neutronics shielding wall 1601C can be positioned such that the beam passing therethrough passes perpendicular to the third neutronics shielding wall 1601C. A fourth neutronics shielding wall 1601D can be positioned between treatment room 1603A and treatment room 1603C to protect the respective rooms and/or their contents from beam interactions.

Distances, dimensions, layout, and angles associated with treatment rooms 1603A, 1603B, 1603C, and neutronics shielding walls 1601A, 1601B, 1601C, and 1601D are configured such that components of system 10 are protected from the neutron back-travel zones 1604A, 1604B, and 1604C.

FIG. 17 illustrates an example beam system usable with embodiments of the present disclosure. In FIG. 17, an example beam system 10 includes an ion source 12, a pre-accelerator system 20 (e.g., or 14), an accelerator 16, and an HEBL 18. Treatment rooms 1703A and 1703B are positioned downstream from accelerator 16 and HEBL 18, and are positioned on a same side of the beamline relative to one another. One or more first magnets 1705A (e.g., quadrupole magnets 52, 72, and/or dipole or bending magnets 56, 58, 2000) are positioned along the beam line such that a beam passing through the beam line intended for treatment room 1703A is steered at a first angle 1702A toward treatment room 1703A. A first neutronics shielding wall 1701A is positioned between the one or more first magnets 1705A and the treatment room 1703A. As a result of the beam encountering first neutronics shielding wall 1701A and/or treatment room 1703A, neutrons can travel away from first treatment room 1703A and/or first neutronics shielding wall 1701A according to a first neutron back-travel zone 1704A depicted in dashed lines.

One or more second magnets 1705B (e.g., quadrupole magnets 52, 72, and/or dipole or bending magnets 56, 58, 2000) are positioned downstream of the one or more first magnets 1705A, and such that the beam is steered at a second angle 1702B toward treatment room 1703B. A second neutronics shielding wall 1701B is positioned between the one or more second magnets 1705B and treatment room 1703B. As a result of the beam encountering second neutronics shielding wall 1701B and/or treatment room 1703B, neutrons can travel away from treatment room 1703B and/or second neutronics shielding wall 1701B according to a second neutron back-travel zone 1704B depicted in dashed lines.

In embodiments, angle 1702A can be 90°+/−5°, or other angles as appropriate for the specific implementation, and angle 1702B can be 90°+/−5°, or other angles as appropriate for the specific implementation. The first neutronics shielding wall 1701A can be positioned such that a beam passing therethrough passes perpendicular to the first neutronics shielding wall 1701A. The second neutronics shielding wall 1701B can be positioned such that the beam passing therethrough passes perpendicular to the second neutronics shielding wall 1701B. A third neutronics shielding wall 1701C can be positioned between treatment room 1703A and treatment room 1703B to protect the respective rooms and/or their contents from beam interactions.

Distances, dimensions, layout, and angles associated with treatment rooms 1703A, 1703B, and neutronics shielding walls 1701A, 1701B, and 1701C are configured such that components of system 10 are protected from the neutron back-travel zones 1704A, 1704B.

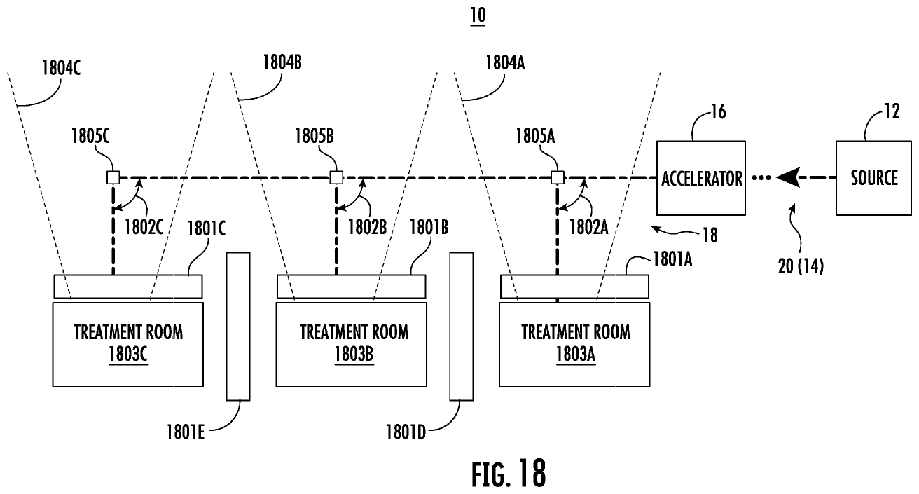

FIG. 18 illustrates an example beam system usable with embodiments of the present disclosure. In FIG. 18, an example beam system 10 includes an ion source 12, a pre-accelerator system 20 (e.g., or 14), an accelerator 16, and an HEBL 18. Treatment rooms 1803A, 1803B, and 1803C are positioned downstream from accelerator 16 and HEBL 18, and are positioned on a same side of the beamline relative to one another. One or more first magnets 1805A (e.g., quadrupole magnets 52, 72, and/or dipole or bending magnets 56, 58, 2000) are positioned along the beam line such that a beam passing through the beam line intended for treatment room 1803A is steered at a first angle 1802A toward treatment room 1803A. A first neutronics shielding wall 1801A is positioned between the one or more first magnets 1805A and the treatment room 1803A. As a result of the beam encountering first neutronics shielding wall 1801A and/or treatment room 1803A, neutrons can travel away from first treatment room 1803A and/or first neutronics shielding wall 1801A according to a first neutron back-travel zone 1804A depicted in dashed lines.

One or more second magnets 1805B (e.g., quadrupole magnets 52, 72, and/or dipole or bending magnets 56, 58, 2000) are positioned downstream of the one or more first magnets 1805A, and such that the beam is steered at a second angle 1802B toward treatment room 1803B. A second neutronics shielding wall 1801B is positioned between the one or more second magnets 1805B and treatment room 1803B. As a result of the beam encountering second neutronics shielding wall 1801B and/or treatment room 1803B, neutrons can travel away from treatment room 1803B and/or second neutronics shielding wall 1801B according to a second neutron back-travel zone 1804B depicted in dashed lines.

One or more third magnets 1805C (e.g., quadrupole magnets 52, 72, and/or dipole or bending magnets 56, 58, 2000) are positioned downstream of the one or more second magnets 1805B, and such that the beam is steered at a third angle 1802C toward treatment room 1803C. A third neutronics shielding wall 1801C is positioned between the one or more third magnets 1805C and treatment room 1803C. As a result of the beam encountering third neutronics shielding wall 1801C and/or treatment room 1803C, neutrons can travel away from treatment room 1803C and/or third neutronics shielding wall 1801C according to a third neutron back-travel zone 1804C depicted in dashed lines.

In embodiments, angle 1802A can be 90°+/−5°, or other angles as appropriate for the specific implementation, angle 1802B can be 90°+/−5°, or other angles as appropriate for the specific implementation, and angle 1802C can be 90°+/−5°, or other angles as appropriate for the specific implementation. The first neutronics shielding wall 1801A can be positioned such that a beam passing therethrough passes perpendicular to the first neutronics shielding wall 1801A. The second neutronics shielding wall 1801B can be positioned such that the beam passing therethrough passes perpendicular to the second neutronics shielding wall 1801B. The third neutronics shielding wall 1801C can be positioned such that the beam passing therethrough passes perpendicular to the third neutronics shielding wall 1801C. A fourth neutronics shielding wall 1801D can be positioned between treatment room 1803A and treatment room 1803B to protect the respective rooms and/or their contents from beam interactions. A fifth neutronics shielding wall 1801E can be positioned between treatment room 1803B and treatment room 1803C to protect the respective rooms and/or their contents from beam interactions.

Distances, dimensions, layout, and angles associated with treatment rooms 1803A, 1803B, 1803C, and neutronics shielding walls 1801A, 1801B, 1801C, 1801D, and 1801E are configured such that components of system 10 are protected from the neutron back-travel zones 1804A, 1804B, and 1804C.

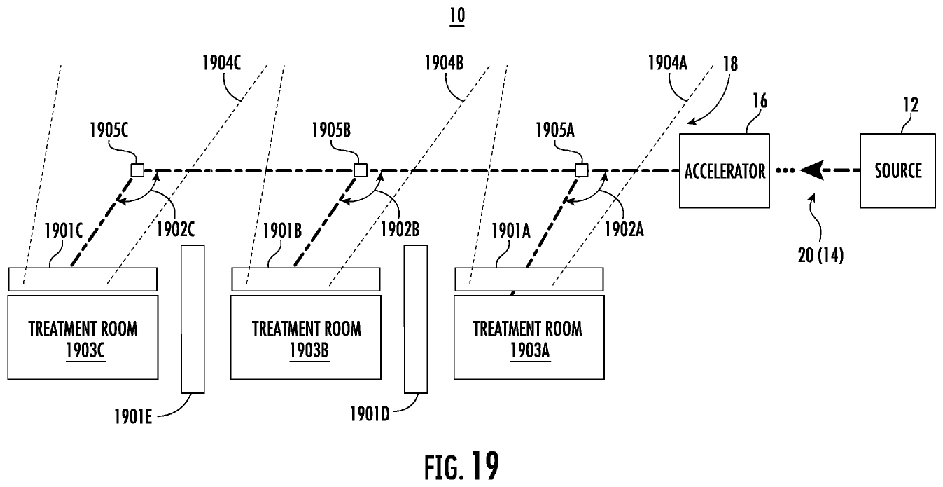

FIG. 19 illustrates an example beam system usable with embodiments of the present disclosure. In FIG. 19, an example beam system 10 includes an ion source 12, a pre-accelerator system 20 (e.g., or 14), an accelerator 16, and an HEBL 18. Treatment rooms 1903A, 1903B, and 1903C are positioned downstream from accelerator 16 and HEBL 18, and are positioned on a same side of the beamline relative to one another. One or more first magnets 1905A (e.g., quadrupole magnets 52, 72, and/or dipole or bending magnets 56, 58, 2000) are positioned along the beam line such that a beam passing through the beam line intended for treatment room 1903A is steered at a first angle 1902A toward treatment room 1903A. A first neutronics shielding wall 1901A is positioned between the one or more first magnets 1905A and the treatment room 1903A. As a result of the beam encountering first neutronics shielding wall 1901A and/or treatment room 1903A, neutrons can travel away from first treatment room 1903A and/or first neutronics shielding wall 1901A according to a first neutron back-travel zone 1904A depicted in dashed lines.

One or more second magnets 1905B (e.g., quadrupole magnets 52, 72, and/or dipole or bending magnets 56, 58, 2000) are positioned downstream of the one or more first magnets 1905A, and such that the beam is steered at a second angle 1902B toward treatment room 1903B. A second neutronics shielding wall 1901B is positioned between the one or more second magnets 1905B and treatment room 1903B. As a result of the beam encountering second neutronics shielding wall 1901B and/or treatment room 1903B, neutrons can travel away from treatment room 1903B and/or second neutronics shielding wall 1901B according to a second neutron back-travel zone 1904B depicted in dashed lines.

One or more third magnets 1905C (e.g., quadrupole magnets 52, 72, and/or dipole or bending magnets 56, 58, 2000) are positioned downstream of the one or more second magnets 1905B, and such that the beam is steered at a third angle 1902C toward treatment room 1903C. A third neutronics shielding wall 1901C is positioned between the one or more third magnets 1905C and treatment room 1903C. As a result of the beam encountering third neutronics shielding wall 1901C and/or treatment room 1903C, neutrons can travel away from treatment room 1903C and/or third neutronics shielding wall 1901C according to a third neutron back-travel zone 1904C depicted in dashed lines.

In embodiments, angle 1902A can be 120°+/−5°, or other angles as appropriate for the specific implementation, angle 1902B can be 120°+/−5°, or other angles as appropriate for the specific implementation, and angle 1902C can be 120°+/−5°, or other angles as appropriate for the specific implementation. The first neutronics shielding wall 1901A can be positioned such that a beam passing therethrough passes perpendicular to the first neutronics shielding wall 1901A. The second neutronics shielding wall 1901B can be positioned such that the beam passing therethrough passes perpendicular to the second neutronics shielding wall 1901B. The third neutronics shielding wall 1901C can be positioned such that the beam passing therethrough passes perpendicular to the third neutronics shielding wall 1901C. A fourth neutronics shielding wall 1901D can be positioned between treatment room 1903A and treatment room 1903B to protect the respective rooms and/or their contents from beam interactions. A fifth neutronics shielding wall 1901E can be positioned between treatment room 1903B and treatment room 1903C to protect the respective rooms and/or their contents from beam interactions.

Distances, dimensions, layout, and angles associated with treatment rooms 1903A, 1903B, 1903C, and neutronics shielding walls 1901A, 1901B, 1901C, 1901D, and 1901E are configured such that components of system 10 are protected from the neutron back-travel zones 1904A, 1904B, and 1904C.

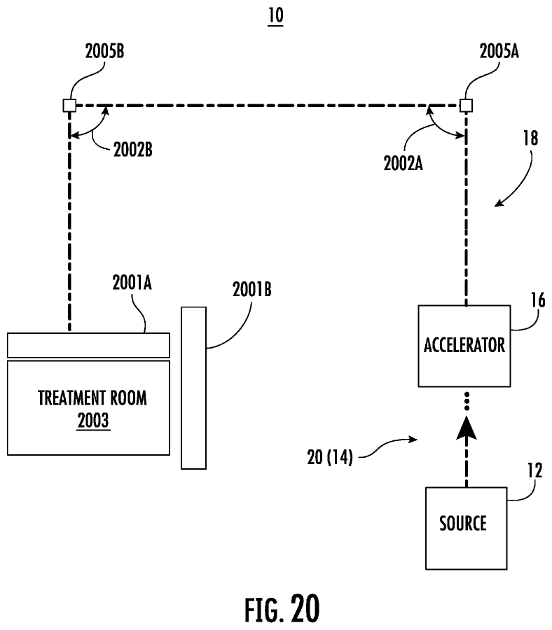

FIG. 20 illustrates an example beam system usable with embodiments of the present disclosure. In FIG. 20, an example beam system 20 includes an ion source 12, a pre-accelerator system 20 (e.g., or 14), an accelerator 16, and an HEBL 18. One or more first magnets 2005A (e.g., quadrupole magnets 52, 72, and/or dipole or bending magnets 56, 58, 2000) are positioned downstream of accelerator 16, such that a beam passing through the system is steered at an angle 2002A toward one or more second magnets 2005B (e.g., quadrupole magnets 52, 72, and/or dipole or bending magnets 56, 58, 2000) positioned downstream of the one or more first magnets 2005A. The one or more second magnets 2005B permit the beam to be steered at an angle 2002B toward treatment room 2003 positioned downstream of the one or more second magnets 2005B. A first neutronics shielding wall 2001A is positioned between the one or more second magnets 2005B and the treatment room 2003. A second neutronics shielding wall 2001B can be positioned between the treatment room 2003 and accelerator 16 (e.g., or other components of system 10 upstream of the treatment room 2003). The first neutronics shielding wall 2001A can be positioned such that a beam passing therethrough passes perpendicular to the first neutronics shielding wall 2001A.

Distances, dimensions, layout, and angles associated with treatment room 2003A and neutronics shielding walls 2001A, 2001B are configured such that components of system 10 are protected from a neutron back-travel zone (not shown) associated with treatment room 2003A.

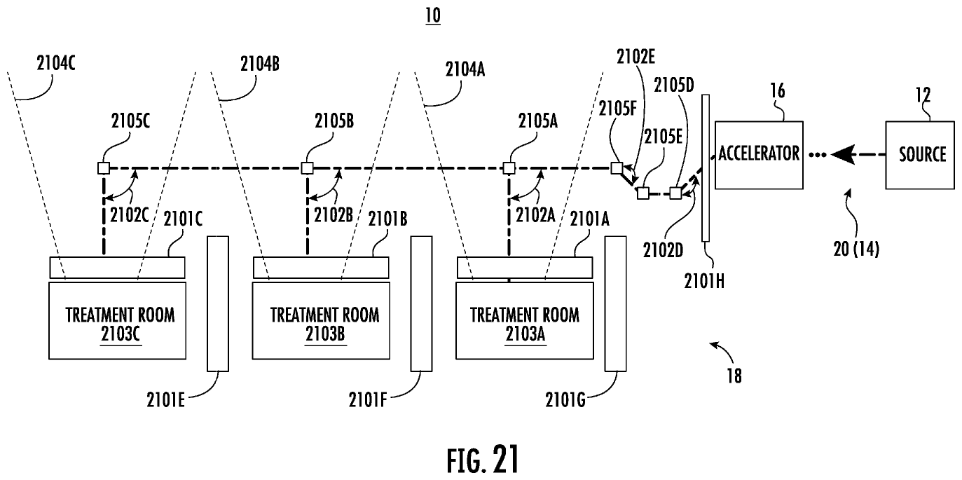

FIG. 21 illustrates an example beam system usable with embodiments of the present disclosure. In FIG. 21, an example beam system 10 includes an ion source 12, a pre-accelerator system 20 (e.g., or 14), an accelerator 16, and an HEBL 18. Treatment rooms 2103A, 2103B, and 2103C are positioned downstream from accelerator 16 and HEBL 18, and are positioned on a same side of the beamline relative to one another. One or more first magnets 2105A (e.g., quadrupole magnets 52, 72, and/or dipole or bending magnets 56, 58, 2000) are positioned along the beam line such that a beam passing through the beam line intended for treatment room 2103A is steered at a first angle 2102A toward treatment room 2103A. A first neutronics shielding wall 2101A is positioned between the one or more first magnets 2105A and the treatment room 2103A. As a result of the beam encountering first neutronics shielding wall 2101A and/or treatment room 2103A, neutrons can travel away from first treatment room 2103A and/or first neutronics shielding wall 2101A according to a first neutron back-travel zone 2104A depicted in dashed lines.

One or more second magnets 2105B (e.g., quadrupole magnets 52, 72, and/or dipole or bending magnets 56, 58, 2000) are positioned downstream of the one or more first magnets 2105A, and such that the beam is steered at a second angle 2102B toward treatment room 2103B. A second neutronics shielding wall 2101B is positioned between the one or more second magnets 2105B and treatment room 2103B. As a result of the beam encountering second neutronics shielding wall 2101B and/or treatment room 2103B, neutrons can travel away from treatment room 2103B and/or second neutronics shielding wall 2101B according to a second neutron back-travel zone 2104B depicted in dashed lines.

One or more third magnets 2105C (e.g., quadrupole magnets 52, 72, and/or dipole or bending magnets 56, 58, 2000) are positioned downstream of the one or more second magnets 2105B, and such that the beam is steered at a third angle 2102C toward treatment room 2103C. A third neutronics shielding wall 2101C is positioned between the one or more third magnets 2105C and treatment room 2103C. As a result of the beam encountering third neutronics shielding wall 2101C and/or treatment room 2103C, neutrons can travel away from treatment room 2103C and/or third neutronics shielding wall 2101C according to a third neutron back-travel zone 2104C depicted in dashed lines.

In embodiments, angle 2102A can be 90°+/−5°, or other angles as appropriate for the specific implementation, angle 2102B can be 90°+/−5°, or other angles as appropriate for the specific implementation, and angle 2102C can be 90°+/−5°, or other angles as appropriate for the specific implementation. The first neutronics shielding wall 2101A can be positioned such that a beam passing therethrough passes perpendicular to the first neutronics shielding wall 2101A. The second neutronics shielding wall 2101B can be positioned such that the beam passing therethrough passes perpendicular to the second neutronics shielding wall 2101B. The third neutronics shielding wall 2101C can be positioned such that the beam passing therethrough passes perpendicular to the third neutronics shielding wall 2101C. A fourth neutronics shielding wall 2101E can be positioned between treatment room 2103C and treatment room 2103B to protect the respective rooms and/or their contents from beam interactions. A fifth neutronics shielding wall 2101F can be positioned between treatment room 2103B and treatment room 2103A to protect the respective rooms and/or their contents from beam interactions. A sixth neutronics shielding wall 2101G can be positioned between treatment room 2103A and accelerator 16 (or other components of system 10) to protect the room and components from beam interactions.

In embodiments, one or more fourth magnets 2105D, one or more fifth magnets 2105E, and one or more sixth magnets 2105F can be positioned downstream of accelerator 16 and upstream of one or more first magnets 2105A. The one or more fourth magnets 2105D, one or more fifth magnets 2105E, and one or more sixth magnets 2105F permit a travel path for a beam passing therethrough such that neutron back-travel that may otherwise damage accelerator 16 is redirected or eliminated. A seventh neutronics shielding wall 2101H can also be positioned between the one or more fourth magnets 2105D and accelerator 16 for additional protection of the accelerator 16 and upstream components of system 10.

Distances, dimensions, layout, and angles associated with treatment rooms 2103A, 2103B, 2103C, and neutronics shielding walls 2101A, 2101B, 2101C, 2101E, 2101F, 2101G, 2101H are configured such that components of system 10 are protected from the neutron back-travel zones 2104A, 2104B, and 2104C.

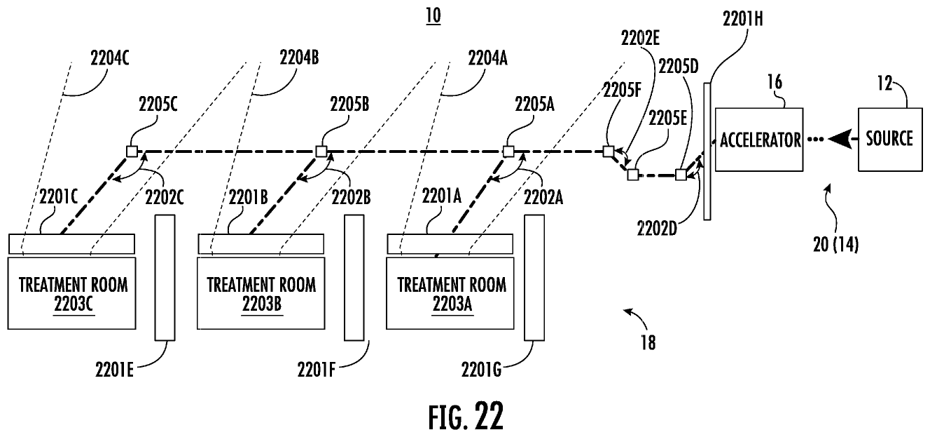

FIG. 22 illustrates an example beam system usable with embodiments of the present disclosure. In FIG. 22, an example beam system 10 includes an ion source 12, a pre-accelerator system 20 (e.g., or 14), an accelerator 16, and an HEBL 18. Treatment rooms 2203A, 2203B, and 2203C are positioned downstream from accelerator 16 and HEBL 18, and are positioned on a same side of the beamline relative to one another. One or more first magnets 2205A (e.g., quadrupole magnets 52, 72, and/or dipole or bending magnets 56, 58, 2000) are positioned along the beam line such that a beam passing through the beam line intended for treatment room 2203A is steered at a first angle 2202A toward treatment room 2203A. A first neutronics shielding wall 2201A is positioned between the one or more first magnets 2205A and the treatment room 2203A. As a result of the beam encountering first neutronics shielding wall 2201A and/or treatment room 2203A, neutrons can travel away from first treatment room 2203A and/or first neutronics shielding wall 2201A according to a first neutron back-travel zone 2204A depicted in dashed lines.

One or more second magnets 2205B (e.g., quadrupole magnets 52, 72, and/or dipole or bending magnets 56, 58, 2000) are positioned downstream of the one or more first magnets 2205A, and such that the beam is steered at a second angle 2202B toward treatment room 2203B. A second neutronics shielding wall 2201B is positioned between the one or more second magnets 2205B and treatment room 2203B. As a result of the beam encountering second neutronics shielding wall 2201B and/or treatment room 2203B, neutrons can travel away from treatment room 2203B and/or second neutronics shielding wall 2201B according to a second neutron back-travel zone 2204B depicted in dashed lines.

One or more third magnets 2205C (e.g., quadrupole magnets 52, 72, and/or dipole or bending magnets 56, 58, 2000) are positioned downstream of the one or more second magnets 2205B, and such that the beam is steered at a third angle 2202C toward treatment room 2203C. A third neutronics shielding wall 2201C is positioned between the one or more third magnets 2205C and treatment room 2203C. As a result of the beam encountering third neutronics shielding wall 2201C and/or treatment room 2203C, neutrons can travel away from treatment room 2203C and/or third neutronics shielding wall 2201C according to a third neutron back-travel zone 2204C depicted in dashed lines.

In embodiments, angle 2202A can be 120°+/−5°, or other angles as appropriate for the specific implementation, angle 2202B can be 120°+/−5°, or other angles as appropriate for the specific implementation, and angle 2202C can be 120°+/−5°, or other angles as appropriate for the specific implementation. The first neutronics shielding wall 2201A can be positioned such that a beam passing therethrough passes perpendicular to the first neutronics shielding wall 2201A. The second neutronics shielding wall 2201B can be positioned such that the beam passing therethrough passes perpendicular to the second neutronics shielding wall 2201B. The third neutronics shielding wall 2201C can be positioned such that the beam passing therethrough passes perpendicular to the third neutronics shielding wall 2201C. A fourth neutronics shielding wall 2201E can be positioned between treatment room 2203C and treatment room 2203B to protect the respective rooms and/or their contents from beam interactions. A fifth neutronics shielding wall 2201F can be positioned between treatment room 2203B and treatment room 2203A to protect the respective rooms and/or their contents from beam interactions. A sixth neutronics shielding wall 2201G can be positioned between treatment room 2203A and accelerator 16 (or other components of system 10) to protect the room and components from beam interactions.

In embodiments, one or more fourth magnets 2205D, one or more fifth magnets 2205E, and one or more sixth magnets 2205F can be positioned downstream of accelerator 16 and upstream of one or more first magnets 2205A. The one or more fourth magnets 2205D, one or more fifth magnets 2205E, and one or more sixth magnets 2205F permit a travel path for a beam passing therethrough such that neutron back-travel that may otherwise damage accelerator 16 is redirected or eliminated. A seventh neutronics shielding wall 2201H can also be positioned between the one or more fourth magnets 2205D and accelerator 16 for additional protection of the accelerator 16 and upstream components of system 10.

Distances, dimensions, layout, and angles associated with treatment rooms 2203A, 2203B, 2203C, and neutronics shielding walls 2201A, 2201B, 2201C, 2201E, 2201F, 2201G, 2201H are configured such that components of system 10 are protected from the neutron back-travel zones 2204A, 2204B, and 2204C.

Various aspects of the present subject matter are set forth below, in review of, and/or in supplementation to, the embodiments described thus far, with the emphasis here being on the interrelation and interchangeability of the following embodiments. In other words, an emphasis is on the fact that each feature of the embodiments can be combined with each and every other feature unless explicitly stated otherwise or logically implausible.

In some embodiments, a method of controlling a travel path of a beam includes propagating a beam along a first path from an entry point of a dipole magnet through a non-gradient portion of the dipole magnet such that the beam bends toward a first beam travel path of multiple beam travel paths of the dipole magnet and continues along the first beam travel path through a gradient portion of the dipole magnet.

In some of these embodiments, the gradient portion of the dipole magnet is configured to focus the beam.

In some of these embodiments, the method further includes directing the beam to the downstream target using a beamline such that particles from the beam collide with the downstream target and generate neutrons upon impacting a neutron generation region of the downstream target.

In some of these embodiments, the method further includes applying a first current to a pair of windings of the dipole magnet such that a first magnetic field of the non-gradient portion directs the beam toward the first beam travel path.

In some of these embodiments, the first beam travel path is along a first side of a vertical central line of the dipole magnet. In some of these embodiments, the method further includes applying the first current in a counter-clockwise direction according to a perspective above the beam for a horizontal bend beamline or according to a perspective from a left side of the dipole magnet when facing the downstream target for a vertical bend beamline.

In some of these embodiments, the first beam travel path is along a second side of a vertical central line of the dipole magnet. In some of these embodiments, the method further includes applying the first current in a clockwise direction according to a perspective above the beam for a horizontal bend beamline or according to a perspective from a left side of the dipole magnet when facing the downstream target for a vertical bend beamline.

In some of these embodiments, the first current includes a current level configured to influence a magnitude of a bend in a direction of travel of the beam toward the first beam travel path.

In some of these embodiments, each beam travel path of the multiple beam travel paths is associated with the gradient portion of the dipole magnet.

In some of these embodiments, the beam is propagated along the first path only until the beam bends enough toward the first beam travel path without being substantially influenced by other beam travel paths of the multiple beam travel paths of the dipole magnet. In some of these embodiments, the entry point of the dipole magnet includes one or more chamfers configured to create a gradient in a direction of the beam without creating a gradient transverse to the direction of the beam.

In some of these embodiments, the first beam travel path is one or more of −90, −45, 0, +45, or +90 degrees from a vertical central line of the dipole magnet.

In some of these embodiments, when the first beam travel path is one of −90 or −45 degrees from the vertical central line, the other beam travel paths of the multiple beam travel paths are one or more of +45 degrees, +90 degrees, or in a range between +45 and +90 degrees from the vertical central line.

In some of these embodiments, when the first beam travel path is one of +90 or +45 degrees from the vertical central line, the other beam travel paths of the multiple beam travel paths are one or more of −45 degrees, −90 degrees, or in a range between −45 and −90 degrees from the vertical central line.

In some of these embodiments, the non-gradient portion of the dipole magnet includes a non-gradient top pole face and a non-gradient bottom pole face. In some of these embodiments, the non-gradient top pole face and non-gradient bottom pole face are substantially parallel and substantially flat relative to one another.

In some of these embodiments, the gradient portion of the dipole magnet includes a gradient top pole face and a gradient bottom pole face. In some of these embodiments, the gradient top pole face and the gradient bottom pole face are angled relative to one another.

In some of these embodiments, the gradient portion is angled relative to the non-gradient portion.

In some of these embodiments, propagating the beam along the first path includes producing a uniform magnetic field within the non-gradient portion of the dipole magnet.

In some of these embodiments, propagating the beam through the gradient portion of the dipole magnet includes producing a non-uniform magnetic field within the gradient portion of the dipole magnet.

In some embodiments, a dipole magnet includes a non-gradient portion including a non-gradient top pole face and a non-gradient bottom pole face that are parallel relative to one another. In some of these embodiments, the dipole magnet further includes a gradient portion including a gradient top pole face and a gradient bottom pole face that are angled relative to the non-gradient portion and to one another. In some of these embodiments, the gradient portion includes multiple beam travel paths.

In some of these embodiments, a first beam travel path of the multiple beam travel paths is along a first side of a vertical central line of the dipole magnet.

In some of these embodiments, a second beam travel path of the multiple beam travel paths is along a second side of the vertical central line of the dipole magnet.

In some of these embodiments, an entry point of the dipole magnet includes one or more chamfers configured to create a gradient in a direction of the beam without creating a gradient transverse to the direction of the beam.

In some of these embodiments, an exit point of one or more of the one or more beam travel paths includes one or more chamfers.

In some of these embodiments, a first beam travel path of the multiple beam travel paths is one or more of −90, −45, 0, +45, or +90 degrees from a vertical central line of the dipole magnet.

In some of these embodiments, when the first beam travel path is one of −90 or −45 degrees from the vertical central line, other beam travel paths of the multiple beam travel paths are one or more of +45 degrees, +90 degrees, or in a range between +45 and +90 degrees from the vertical central line.

In some of these embodiments, when the first beam travel path is one of +90 or +45 degrees from the vertical central line, other beam travel paths of the multiple beam travel paths are one or more of −45 degrees, −90 degrees, or in a range between −45 and −90 degrees from the vertical central line.

In some of these embodiments, the dipole magnet further includes a core structure having a first pole portion and a second pole portion. In some of these embodiments, the non-gradient top pole face and the gradient top pole face are on the first pole portion. In some of these embodiments, the non-gradient bottom pole face and the gradient bottom pole face are on the second pole portion.

In some of these embodiments, the dipole magnet further includes a first winding around the first pole portion, and a second winding around the second pole portion.

In some of these embodiments, the core structure is laminated. In some of these embodiments, the laminated core structure is parallel to a magnetic field of the non-gradient portion.

In some of these embodiments, the dipole magnet further includes a void separating a first travel path of the gradient portion and a second travel path of the gradient portion.

In some of these embodiments, the dipole magnet further includes a void separating a first travel path of the gradient portion and a second travel path of the gradient portion such that a straight beam exit is established.

In some embodiments, a beam system includes an accelerator, and a beamline extending from the accelerator to a target. In some of these embodiments, the beamline includes a dipole magnet configured to propagate a beam along a first path from an entry point of the dipole magnet through a non-gradient portion of the dipole magnet such that the beam bends toward a first beam travel path of multiple beam travel paths of the dipole magnet and continues along the first beam travel path through a gradient portion of the dipole magnet toward the target.

In some of these embodiments, the beamline is configured to focus the beam toward the target with the dipole magnet and without a quadrupole magnet.

In some embodiments, a beam system includes an accelerator, and a beamline extending from the accelerator to a first treatment room. In some of these embodiments, the beamline includes a first magnet configured to steer a beam passing through the beamline at a first angle and a first neutronics shielding wall positioned between the first magnet and the first treatment room.

In some of these embodiments, the first angle is one of 45°, 60°, 90°, or 120°.

In some of these embodiments, the beamline further includes a second neutronics shielding wall positioned between the accelerator and the first magnet. In some of these embodiments, the beamline further includes a second magnet, positioned between the first magnet and the first neutronics shielding wall. In some of these embodiments, the second magnet is configured to steer the beam passing through the beamline at a second angle toward the first treatment room. In some of these embodiments, the second angle is one of 45°, 60°, 90°, or 120°.

In some of these embodiments, the first neutronics shielding wall is positioned perpendicular to an exit of the accelerator. In some of these embodiments, the first neutronics shielding wall is positioned perpendicular to an axis of a beam passing therethrough.

In some of these embodiments, the second neutronics shielding wall is positioned perpendicular to an exit of the accelerator. In some of these embodiments, the second neutronics shielding wall is positioned perpendicular to an axis of a beam passing therethrough. In some of these embodiments, the first neutronics shielding wall and the second neutronics shielding wall are parallel to each other. In some of these embodiments, the first neutronics shielding wall and the second neutronics shielding wall are separated by a distance.

In some of these embodiments, the first magnet is configured to steer the beam to a second treatment room at a second angle. In some of these embodiments, the second treatment room is downstream of the first treatment room. In some of these embodiments, the beamline further includes a second neutronics shielding wall positioned between the first magnet and the second treatment room.

In some of these embodiments, the first magnet includes a dipole magnet. In some of these embodiments, the dipole magnet includes a non-gradient portion including a non-gradient top pole face and a non-gradient bottom pole face that are parallel relative to one another, and a gradient portion including a gradient top pole face and a gradient bottom pole face that are angled relative to the non-gradient portion and to one another. In some of these embodiments, the gradient portion includes multiple beam travel paths.

In some of these embodiments, the beamline further includes a second magnet, downstream of the first magnet, configured to steer the beam to a second treatment room at a second angle. In some of these embodiments, the beamline further includes a second neutronics shielding wall positioned between the second magnet and the second treatment room. In some of these embodiments, the second angle is one of 45°, 60°, 90°, or 120°.

In some of these embodiments, the beamline further includes a third magnet, downstream of the third magnet, configured to steer the beam to a third treatment room at a third angle. In some of these embodiments, the beamline further includes a third neutronics shielding wall positioned between the third magnet and the third treatment room. In some of these embodiments, the third angle is one of 45°, 60°, 90°, or 120°.

In some of these embodiments, the beamline further includes one or more additional neutronics shielding walls positioned between any of the first treatment room, second treatment room, third treatment room, or beamline components.

In some of these embodiments, the beamline further includes one or more additional magnets positioned downstream of the accelerator and prior to the first magnet or second magnet. In some of these embodiments, the one or more additional magnets configure a path along which a beam may travel such that neutron back-travel exposure to the accelerator is minimized or reduced.

In some of these embodiments, the first neutronics shielding wall includes shielding configured to protect beamline components from neutron back-travel exposure associated with a beam passing therethrough.

In some of these embodiments, a magnet is one or more of a quadrupole magnet or a dipole magnet.

In some of these embodiments, the beam system is configured for use in boron neutron capture therapy (BNCT).

In some of these embodiments, the beamline extends into any of the first, second or third treatment rooms. In some of these embodiments, a neutron target assembly is positioned at an end of the beamline. In some of these embodiments, the neutron target assembly is configured to house a target. In some of these embodiments, the neutron target assembly includes a beam shaping assembly that is integrated into a neutronic shielding wall. In some of these embodiments, the target includes lithium.

In some embodiments, a method of controlling a travel path of a beam includes propagating a beam along a first path from an accelerator through a first magnet such that the beam bends toward a first treatment room at a first angle. In some of these embodiments, the beam propagates through a first neutronics shielding wall prior to entering the treatment room. In some of these embodiments, the method uses a beam system according to any of the foregoing example embodiments.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

As used herein and in the appended claims, the singular forms “a,” “an,” and “the” include plural referents unless the context clearly dictates otherwise.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

1. A method of controlling a travel path of a beam, the method comprising:
propagating a beam along a first path from an entry point of a dipole magnet through a non-gradient portion of the dipole magnet such that the beam bends toward a first beam travel path of a plurality of beam travel paths of the dipole magnet and continues along the first beam travel path through a gradient portion of the dipole magnet.

2. The method of claim 1, wherein the gradient portion of the dipole magnet is configured to focus the beam.

3. The method of claim 1, further comprising:
directing the beam to the downstream target using a beamline such that particles from the beam collide with the downstream target and generate neutrons upon impacting a neutron generation region of the downstream target.

4. The method of claim 1, further comprising applying a first current to a pair of windings of the dipole magnet such that a first magnetic field of the non-gradient portion directs the beam toward the first beam travel path.

5. The method of claim 4, wherein the first beam travel path is along a first side of a vertical central line of the dipole magnet, the method further comprising applying the first current in a counter-clockwise direction according to a perspective above the beam for a horizontal bend beamline or according to a perspective from a left side of the dipole magnet when facing the downstream target for a vertical bend beamline.

6. The method of claim 4, wherein the first beam travel path is along a second side of a vertical central line of the dipole magnet, the method further comprising applying the first current in a clockwise direction according to a perspective above the beam for a horizontal bend beamline or according to a perspective from a left side of the dipole magnet when facing the downstream target for a vertical bend beamline.

7-20. (canceled)

21. A dipole magnet, comprising:
a non-gradient portion comprising a non-gradient top pole face and a non-gradient bottom pole face that are parallel relative to one another; and
a gradient portion comprising a gradient top pole face and a gradient bottom pole face that are angled relative to the non-gradient portion and to one another, wherein the gradient portion comprises a plurality of beam travel paths.

22. The dipole magnet of claim 21, wherein a first beam travel path of the plurality of beam travel paths is along a first side of a vertical central line of the dipole magnet.

23. The dipole magnet of claim 22, wherein a second beam travel path of the plurality of beam travel paths is along a second side of the vertical central line of the dipole magnet.

24. The dipole magnet of claim 21, wherein an entry point of the dipole magnet comprises one or more chamfers configured to create a gradient in a direction of the beam without creating a gradient transverse to the direction of the beam.

25. The dipole magnet of claim 21, wherein an exit point of one or more of the one or more beam travel paths comprises one or more chamfers.

26. The dipole magnet of claim 21, wherein a first beam travel path of the plurality of beam travel paths is one or more of −90, −45, 0, +45, or +90 degrees from a vertical central line of the dipole magnet.

27. The dipole magnet of claim 26, wherein, when the first beam travel path is one of −90 or −45 degrees from the vertical central line, other beam travel paths of the plurality of beam travel paths are one or more of +45 degrees, +90 degrees, or in a range between +45 and +90 degrees from the vertical central line.

28. The dipole magnet of claim 26, wherein, when the first beam travel path is one of +90 or +45 degrees from the vertical central line, other beam travel paths of the plurality of beam travel paths are one or more of −45 degrees, −90 degrees, or in a range between −45 and −90 degrees from the vertical central line.

29. The dipole magnet of claim 21, further comprising a core structure having a first pole portion and a second pole portion, wherein the non-gradient top pole face and the gradient top pole face are on the first pole portion, and wherein the non-gradient bottom pole face and the gradient bottom pole face are on the second pole portion.

30. The dipole magnet of claim 29, further comprising:
a first winding around the first pole portion; and
a second winding around the second pole portion.

31. The dipole magnet of claim 29, wherein the core structure is laminated.

32. The dipole magnet of claim 31, wherein the laminated core structure is parallel to a magnetic field of the non-gradient portion.

33. The dipole magnet of claim 21, further comprising a void separating a first travel path of the gradient portion and a second travel path of the gradient portion.

34. The dipole magnet of claim 21, further comprising a void separating a first travel path of the gradient portion and a second travel path of the gradient portion such that a straight beam exit is established.

35-67. (canceled)
